SEQUENCE LISTING (16 sequences)

SEQ ID NO: 1 - protein, 719 aa, Geobacillus stearothermophilus
  SIGNAL 1..33
MKKKTLSLFVGLMLLIGLLFSGSLPYNPNAAEASSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 2 - protein, 686 aa, Geobacillus stearothermophilus
SSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 3 - protein, 686 aa, synthetic construct
SSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNLTDPAGFSLADLSQENGTIAQYLTDAAVQLVAHGADGLRIDAVKHFNSGFSKSLADKLYQKKDIFLVGEWYGDGPGTANHLEKVRYANNSGVNVLDFDLNPVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 4 - protein, 686 aa, synthetic construct
  VARIANT 1: note = is different from S, in an embodiment is G or T
  VARIANT 2: note = is different from S, in an embodiment is G or T
  VARIANT 188: note = is F or L
  VARIANT 261: note = is D or G
  VARIANT 288: note = is T or P
  VARIANT 676: note = is A or V
XXSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNXTDPAGFSLADLSQENGTIAQYLTDAAVQLVAHGADGLRIDAVKHFNSGFSKSLADKLYQKKDIFLVGEWYGDXPGTANHLEKVRYANNSGVNVLDFDLNXVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGXTGNITVTWQN

SEQ ID NO: 5 - protein, 686 aa, synthetic construct
GSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 6 - protein, 686 aa, synthetic construct
TSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 7 - protein, 686 aa, synthetic construct
GSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNLTDPAGFSLADLSQENGTIAQYLTDAAVQLVAHGADGLRIDAVKHFNSGFSKSLADKLYQKKDIFLVGEWYGDGPGTANHLEKVRYANNSGVNVLDFDLNPVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 8 - protein, 686 aa, synthetic construct
TSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNLTDPAGFSLADLSQENGTIAQYLTDAAVQLVAHGADGLRIDAVKHFNSGFSKSLADKLYQKKDIFLVGEWYGDGPGTANHLEKVRYANNSGVNVLDFDLNPVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN

SEQ ID NO: 9 - protein, 686 aa, synthetic construct
GSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNLTDPAGFSLADLSQENGTIAQYLTDAAVQLVAHGADGLRIDAVKHFNSGFSKSLADKLYQKKDIFLVGEWYGDGPGTANHLEKVRYANNSGVNVLDFDLNPVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGVTGNITVTWQN

SEQ ID NO: 10 - DNA, 2064 bp, synthetic construct
  source 1..2064: mol_type = other DNA
atgggttcttctgcttctgtaaaaggtgatgtcatctaccaaatcatcatcgacagattttacgatggtgataccactaacaacaacccagctaaatcttacggtttgtacgatccaactaagtccaaatggaaaatgtattggggtggtgatttggaaggtgtcagacaaaaattgccatacttgaagcaattgggtgttaccactatttggttgtctccagttttggataacttggatactttggctggtactgataacactggttatcatggttattggaccagagacttcaagcaaatcgaagaacattttggtaactggactaccttcgataccttggttaatgatgctcatcaaaacggtatcaaggttatcgttgatttcgttccaaaccattctactccattcaaggctaacgattctacttttgctgaaggtggtgccttgtataacaatggtacttacatgggtaactacttcgatgatgctactaagggttacttccatcataacggtgatatctctaactgggatgatagatatgaagcccaatggaagaatttgactgatccagctggtttttctttggccgatttgtctcaagaaaacggtactattgctcaatacttgactgatgctgctgttcaattggttgctcatggtgctgatggtttgagaattgatgctgttaagcacttcaactccggtttctctaaatctttggctgacaagttgtaccaaaagaaggacattttcttggtcggtgaatggtatggtgatggtccaggtactgctaatcatttggaaaaggttagatacgccaacaactctggtgttaacgttttggatttcgatttgaacccagtcatcagaaacgttttcggtactttcactcaaactatgtacgacttgaacaacatggtcaatcaaaccggtaacgaatacaagtacaaagaaaacttgatcaccttcatcgacaaccacgatatgtctagattcttgtccgttaactccaacaaagccaacttgcatcaagctttggctttcattttgacctcaagaggtactccatctatctactatggtactgaacaatatatggccggtggtaatgatccatacaatagaggtatgatgccagctttcgatacaactactactgccttcaaagaagtttctacattggcaggtttaagaagaaacaacgctgctattcaatacggtactactactcaaagatggatcaacaacgatgtctacatctacgaaagaaagttcttcaacgatgttgttttggtcgccatcaacagaaatacccaatcctcttattccatctccggtttacaaactgctttgccaaatggttcttacgccgattatttgtctggtttgttaggtggtaacggtatctctgtttctaatggttctgttgcttctttcactttggctccaggtgctgtttctgtttggcaatactctacttcagcttctgctccacaaattggttcagttgctccaaatatgggtattccaggtaacgttgttaccattgatggtaagggttttggtactacacaaggtactgttacttttggtggtgttactgctactgttaagtcttggacttccaacagaatcgaagtttacgttcctaatatggctgctggtttgacagatgttaaggttactgccggtggtgtttcttcaaacttgtactcttacaacatcttgtccggtactcaaacctctgttgtttttacagttaagtcagctccaccaactaacttgggtgataagatctatttgaccggtaacattccagaattgggtaattggtctactgatacttctggtgcagttaacaatgctcaaggtccattattggctccaaattacccagattggttctacgttttttctgttccagccggtaagaccattcaattcaagtttttcattaagagagccgacggtacaattcaatgggaaaatggttcaaaccatgttgctactactccaactggtgctactggtaacattactgttacatggcaaaactaa

SEQ ID NO: 11 - DNA, 2064 bp, synthetic construct
  source 1..2064: mol_type = other DNA
atgggttcttctgcttctgtaaaaggtgatgtcatctaccaaatcatcatcgacagattttacgatggtgataccactaacaacaacccagctaaatcttacggtttgtacgatccaactaagtccaaatggaaaatgtattggggtggtgatttggaaggtgtcagacaaaaattgccatacttgaagcaattgggtgttaccactatttggttgtctccagttttggataacttggatactttggctggtactgataacactggttatcatggttattggaccagagacttcaagcaaatcgaagaacattttggtaactggactaccttcgataccttggttaatgatgctcatcaaaacggtatcaaggttatcgttgatttcgttccaaaccattctactccattcaaggctaacgattctacttttgctgaaggtggtgccttgtataacaatggtacttacatgggtaactacttcgatgatgctactaagggttacttccatcataacggtgatatctctaactgggatgatagatatgaagcccaatggaagaatttgactgatccagctggtttttctttggccgatttgtctcaagaaaacggtactattgctcaatacttgactgatgctgctgttcaattggttgctcatggtgctgatggtttgagaattgatgctgttaagcacttcaactccggtttctctaaatctttggctgacaagttgtaccaaaagaaggacattttcttggtcggtgaatggtatggtgatggtccaggtactgctaatcatttggaaaaggttagatacgccaacaactctggtgttaacgttttggatttcgatttgaacccagtcatcagaaacgttttcggtactttcactcaaactatgtacgacttgaacaacatggtcaatcaaaccggtaacgaatacaagtacaaagaaaacttgatcaccttcatcgacaaccacgatatgtctagattcttgtccgttaactccaacaaagccaacttgcatcaagctttggctttcattttgacctcaagaggtactccatctatctactatggtactgaacaatatatggccggtggtaatgatccatacaatagaggtatgatgccagctttcgatacaactactactgccttcaaagaagtttctacattggcaggtttaagaagaaacaacgctgctattcaatacggtactactactcaaagatggatcaacaacgatgtctacatctacgaaagaaagttcttcaacgatgttgttttggtcgccatcaacagaaatacccaatcctcttattccatctccggtttacaaactgctttgccaaatggttcttacgccgattatttgtctggtttgttaggtggtaacggtatctctgtttctaatggttctgttgcttctttcactttggctccaggtgctgtttctgtttggcaatactctacttcagcttctgctccacaaattggttcagttgctccaaatatgggtattccaggtaacgttgttaccattgatggtaagggttttggtactacacaaggtactgttacttttggtggtgttactgctactgttaagtcttggacttccaacagaatcgaagtttacgttcctaatatggctgctggtttgacagatgttaaggttactgccggtggtgtttcttcaaacttgtactcttacaacatcttgtccggtactcaaacctctgttgtttttacagttaagtcagctccaccaactaacttgggtgataagatctatttgaccggtaacattccagaattgggtaattggtctactgatacttctggtgcagttaacaatgctcaaggtccattattggctccaaattacccagattggttctacgttttttctgttccagccggtaagaccattcaattcaagtttttcattaagagagccgacggtacaattcaatgggaaaatggttcaaaccatgttgctactactccaactggtgttactggtaacattactgttacatggcaaaactaa

SEQ ID NO: 12 - DNA, 2064 bp, synthetic construct
  source 1..2064: mol_type = other DNA
atgtcttcttctgcttctgtaaaaggtgatgtcatctaccaaatcatcatcgacagattttacgatggtgataccactaacaacaacccagctaaatcttacggtttgtacgatccaactaagtccaaatggaaaatgtattggggtggtgatttggaaggtgtcagacaaaaattgccatacttgaagcaattgggtgttaccactatttggttgtctccagttttggataacttggatactttggctggtactgataacactggttatcatggttattggaccagagacttcaagcaaatcgaagaacattttggtaactggactaccttcgataccttggttaatgatgctcatcaaaacggtatcaaggttatcgttgatttcgttccaaaccattctactccattcaaggctaacgattctacttttgctgaaggtggtgccttgtataacaatggtacttacatgggtaactacttcgatgatgctactaagggttacttccatcataacggtgatatctctaactgggatgatagatatgaagcccaatggaagaatttcactgatccagctggtttttctttggccgatttgtctcaagaaaacggtactattgctcaatacttgactgatgctgctgttcaattggttgctcatggtgctgatggtttgagaattgatgctgttaagcacttcaactccggtttctctaaatctttggctgacaagttgtaccaaaagaaggacattttcttggtcggtgaatggtatggtgatgatccaggtactgctaatcatttggaaaaggttagatacgccaacaactctggtgttaacgttttggatttcgatttgaacaccgtcatcagaaacgttttcggtactttcactcaaactatgtacgacttgaacaacatggtcaatcaaaccggtaacgaatacaagtacaaagaaaacttgatcaccttcatcgacaaccacgatatgtctagattcttgtccgttaactccaacaaagccaacttgcatcaagctttggctttcattttgacctcaagaggtactccatctatctactatggtactgaacaatatatggccggtggtaatgatccatacaatagaggtatgatgccagctttcgatacaactactactgccttcaaagaagtttctacattggcaggtttaagaagaaacaacgctgctattcaatacggtactactactcaaagatggatcaacaacgatgtctacatctacgaaagaaagttcttcaacgatgttgttttggtcgccatcaacagaaatacccaatcctcttattccatctccggtttacaaactgctttgccaaatggttcttacgccgattatttgtctggtttgttaggtggtaacggtatctctgtttctaatggttctgttgcttctttcactttggctccaggtgctgtttctgtttggcaatactctacttcagcttctgctccacaaattggttcagttgctccaaatatgggtattccaggtaacgttgttaccattgatggtaagggttttggtactacacaaggtactgttacttttggtggtgttactgctactgttaagtcttggacttccaacagaatcgaagtttacgttcctaatatggctgctggtttgacagatgttaaggttactgccggtggtgtttcttcaaacttgtactcttacaacatcttgtccggtactcaaacctctgttgtttttacagttaagtcagctccaccaactaacttgggtgataagatctatttgaccggtaacattccagaattgggtaattggtctactgatacttctggtgcagttaacaatgctcaaggtccattattggctccaaattacccagattggttctacgttttttctgttccagccggtaagaccattcaattcaagtttttcattaagagagccgacggtacaattcaatgggaaaatggttcaaaccatgttgctactactccaactggtgctactggtaacattactgttacatggcaaaactaa

SEQ ID NO: 13 - DNA, 2064 bp, synthetic construct
  source 1..2064: mol_type = other DNA
atgtcttcttctgcttctgtaaaaggtgatgtcatctaccaaatcatcatcgacagattttacgatggtgataccactaacaacaacccagctaaatcttacggtttgtacgatccaactaagtccaaatggaaaatgtattggggtggtgatttggaaggtgtcagacaaaaattgccatacttgaagcaattgggtgttaccactatttggttgtctccagttttggataacttggatactttggctggtactgataacactggttatcatggttattggaccagagacttcaagcaaatcgaagaacattttggtaactggactaccttcgataccttggttaatgatgctcatcaaaacggtatcaaggttatcgttgatttcgttccaaaccattctactccattcaaggctaacgattctacttttgctgaaggtggtgccttgtataacaatggtacttacatgggtaactacttcgatgatgctactaagggttacttccatcataacggtgatatctctaactgggatgatagatatgaagcccaatggaagaatttgactgatccagctggtttttctttggccgatttgtctcaagaaaacggtactattgctcaatacttgactgatgctgctgttcaattggttgctcatggtgctgatggtttgagaattgatgctgttaagcacttcaactccggtttctctaaatctttggctgacaagttgtaccaaaagaaggacattttcttggtcggtgaatggtatggtgatggtccaggtactgctaatcatttggaaaaggttagatacgccaacaactctggtgttaacgttttggatttcgatttgaacccagtcatcagaaacgttttcggtactttcactcaaactatgtacgacttgaacaacatggtcaatcaaaccggtaacgaatacaagtacaaagaaaacttgatcaccttcatcgacaaccacgatatgtctagattcttgtccgttaactccaacaaagccaacttgcatcaagctttggctttcattttgacctcaagaggtactccatctatctactatggtactgaacaatatatggccggtggtaatgatccatacaatagaggtatgatgccagctttcgatacaactactactgccttcaaagaagtttctacattggcaggtttaagaagaaacaacgctgctattcaatacggtactactactcaaagatggatcaacaacgatgtctacatctacgaaagaaagttcttcaacgatgttgttttggtcgccatcaacagaaatacccaatcctcttattccatctccggtttacaaactgctttgccaaatggttcttacgccgattatttgtctggtttgttaggtggtaacggtatctctgtttctaatggttctgttgcttctttcactttggctccaggtgctgtttctgtttggcaatactctacttcagcttctgctccacaaattggttcagttgctccaaatatgggtattccaggtaacgttgttaccattgatggtaagggttttggtactacacaaggtactgttacttttggtggtgttactgctactgttaagtcttggacttccaacagaatcgaagtttacgttcctaatatggctgctggtttgacagatgttaaggttactgccggtggtgtttcttcaaacttgtactcttacaacatcttgtccggtactcaaacctctgttgtttttacagttaagtcagctccaccaactaacttgggtgataagatctatttgaccggtaacattccagaattgggtaattggtctactgatacttctggtgcagttaacaatgctcaaggtccattattggctccaaattacccagattggttctacgttttttctgttccagccggtaagaccattcaattcaagtttttcattaagagagccgacggtacaattcaatgggaaaatggttcaaaccatgttgctactactccaactggtgctactggtaacattactgttacatggcaaaactaa

SEQ ID NO: 14 - DNA, 2064 bp, synthetic construct
  source 1..2064: mol_type = other DNA
atgggttcttctgcttctgtaaaaggtgatgtcatctaccaaatcatcatcgacagattttacgatggtgataccactaacaacaacccagctaaatcttacggtttgtacgatccaactaagtccaaatggaaaatgtattggggtggtgatttggaaggtgtcagacaaaaattgccatacttgaagcaattgggtgttaccactatttggttgtctccagttttggataacttggatactttggctggtactgataacactggttatcatggttattggaccagagacttcaagcaaatcgaagaacattttggtaactggactaccttcgataccttggttaatgatgctcatcaaaacggtatcaaggttatcgttgatttcgttccaaaccattctactccattcaaggctaacgattctacttttgctgaaggtggtgccttgtataacaatggtacttacatgggtaactacttcgatgatgctactaagggttacttccatcataacggtgatatctctaactgggatgatagatatgaagcccaatggaagaatttcactgatccagctggtttttctttggccgatttgtctcaagaaaacggtactattgctcaatacttgactgatgctgctgttcaattggttgctcatggtgctgatggtttgagaattgatgctgttaagcacttcaactccggtttctctaaatctttggctgacaagttgtaccaaaagaaggacattttcttggtcggtgaatggtatggtgatgatccaggtactgctaatcatttggaaaaggttagatacgccaacaactctggtgttaacgttttggatttcgatttgaacaccgtcatcagaaacgttttcggtactttcactcaaactatgtacgacttgaacaacatggtcaatcaaaccggtaacgaatacaagtacaaagaaaacttgatcaccttcatcgacaaccacgatatgtctagattcttgtccgttaactccaacaaagccaacttgcatcaagctttggctttcattttgacctcaagaggtactccatctatctactatggtactgaacaatatatggccggtggtaatgatccatacaatagaggtatgatgccagctttcgatacaactactactgccttcaaagaagtttctacattggcaggtttaagaagaaacaacgctgctattcaatacggtactactactcaaagatggatcaacaacgatgtctacatctacgaaagaaagttcttcaacgatgttgttttggtcgccatcaacagaaatacccaatcctcttattccatctccggtttacaaactgctttgccaaatggttcttacgccgattatttgtctggtttgttaggtggtaacggtatctctgtttctaatggttctgttgcttctttcactttggctccaggtgctgtttctgtttggcaatactctacttcagcttctgctccacaaattggttcagttgctccaaatatgggtattccaggtaacgttgttaccattgatggtaagggttttggtactacacaaggtactgttacttttggtggtgttactgctactgttaagtcttggacttccaacagaatcgaagtttacgttcctaatatggctgctggtttgacagatgttaaggttactgccggtggtgtttcttcaaacttgtactcttacaacatcttgtccggtactcaaacctctgttgtttttacagttaagtcagctccaccaactaacttgggtgataagatctatttgaccggtaacattccagaattgggtaattggtctactgatacttctggtgcagttaacaatgctcaaggtccattattggctccaaattacccagattggttctacgttttttctgttccagccggtaagaccattcaattcaagtttttcattaagagagccgacggtacaattcaatgggaaaatggttcaaaccatgttgctactactccaactggtgctactggtaacattactgttacatggcaaaactaa

SEQ ID NO: 15 - DNA, 2064 bp, synthetic construct
  source 1..2064: mol_type = other DNA
atgacgtcttctgcttctgtaaaaggtgatgtcatctaccaaatcatcatcgacagattttacgatggtgataccactaacaacaacccagctaaatcttacggtttgtacgatccaactaagtccaaatggaaaatgtattggggtggtgatttggaaggtgtcagacaaaaattgccatacttgaagcaattgggtgttaccactatttggttgtctccagttttggataacttggatactttggctggtactgataacactggttatcatggttattggaccagagacttcaagcaaatcgaagaacattttggtaactggactaccttcgataccttggttaatgatgctcatcaaaacggtatcaaggttatcgttgatttcgttccaaaccattctactccattcaaggctaacgattctacttttgctgaaggtggtgccttgtataacaatggtacttacatgggtaactacttcgatgatgctactaagggttacttccatcataacggtgatatctctaactgggatgatagatatgaagcccaatggaagaatttcactgatccagctggtttttctttggccgatttgtctcaagaaaacggtactattgctcaatacttgactgatgctgctgttcaattggttgctcatggtgctgatggtttgagaattgatgctgttaagcacttcaactccggtttctctaaatctttggctgacaagttgtaccaaaagaaggacattttcttggtcggtgaatggtatggtgatgatccaggtactgctaatcatttggaaaaggttagatacgccaacaactctggtgttaacgttttggatttcgatttgaacaccgtcatcagaaacgttttcggtactttcactcaaactatgtacgacttgaacaacatggtcaatcaaaccggtaacgaatacaagtacaaagaaaacttgatcaccttcatcgacaaccacgatatgtctagattcttgtccgttaactccaacaaagccaacttgcatcaagctttggctttcattttgacctcaagaggtactccatctatctactatggtactgaacaatatatggccggtggtaatgatccatacaatagaggtatgatgccagctttcgatacaactactactgccttcaaagaagtttctacattggcaggtttaagaagaaacaacgctgctattcaatacggtactactactcaaagatggatcaacaacgatgtctacatctacgaaagaaagttcttcaacgatgttgttttggtcgccatcaacagaaatacccaatcctcttattccatctccggtttacaaactgctttgccaaatggttcttacgccgattatttgtctggtttgttaggtggtaacggtatctctgtttctaatggttctgttgcttctttcactttggctccaggtgctgtttctgtttggcaatactctacttcagcttctgctccacaaattggttcagttgctccaaatatgggtattccaggtaacgttgttaccattgatggtaagggttttggtactacacaaggtactgttacttttggtggtgttactgctactgttaagtcttggacttccaacagaatcgaagtttacgttcctaatatggctgctggtttgacagatgttaaggttactgccggtggtgtttcttcaaacttgtactcttacaacatcttgtccggtactcaaacctctgttgtttttacagttaagtcagctccaccaactaacttgggtgataagatctatttgaccggtaacattccagaattgggtaattggtctactgatacttctggtgcagttaacaatgctcaaggtccattattggctccaaattacccagattggttctacgttttttctgttccagccggtaagaccattcaattcaagtttttcattaagagagccgacggtacaattcaatgggaaaatggttcaaaccatgttgctactactccaactggtgctactggtaacattactgttacatggcaaaactaa

SEQ ID NO: 16 - DNA, 2064 bp, synthetic construct
  source 1..2064: mol_type = other DNA
atgacgtcttctgcttctgtaaaaggtgatgtcatctaccaaatcatcatcgacagattttacgatggtgataccactaacaacaacccagctaaatcttacggtttgtacgatccaactaagtccaaatggaaaatgtattggggtggtgatttggaaggtgtcagacaaaaattgccatacttgaagcaattgggtgttaccactatttggttgtctccagttttggataacttggatactttggctggtactgataacactggttatcatggttattggaccagagacttcaagcaaatcgaagaacattttggtaactggactaccttcgataccttggttaatgatgctcatcaaaacggtatcaaggttatcgttgatttcgttccaaaccattctactccattcaaggctaacgattctacttttgctgaaggtggtgccttgtataacaatggtacttacatgggtaactacttcgatgatgctactaagggttacttccatcataacggtgatatctctaactgggatgatagatatgaagcccaatggaagaatttgactgatccagctggtttttctttggccgatttgtctcaagaaaacggtactattgctcaatacttgactgatgctgctgttcaattggttgctcatggtgctgatggtttgagaattgatgctgttaagcacttcaactccggtttctctaaatctttggctgacaagttgtaccaaaagaaggacattttcttggtcggtgaatggtatggtgatggtccaggtactgctaatcatttggaaaaggttagatacgccaacaactctggtgttaacgttttggatttcgatttgaacccagtcatcagaaacgttttcggtactttcactcaaactatgtacgacttgaacaacatggtcaatcaaaccggtaacgaatacaagtacaaagaaaacttgatcaccttcatcgacaaccacgatatgtctagattcttgtccgttaactccaacaaagccaacttgcatcaagctttggctttcattttgacctcaagaggtactccatctatctactatggtactgaacaatatatggccggtggtaatgatccatacaatagaggtatgatgccagctttcgatacaactactactgccttcaaagaagtttctacattggcaggtttaagaagaaacaacgctgctattcaatacggtactactactcaaagatggatcaacaacgatgtctacatctacgaaagaaagttcttcaacgatgttgttttggtcgccatcaacagaaatacccaatcctcttattccatctccggtttacaaactgctttgccaaatggttcttacgccgattatttgtctggtttgttaggtggtaacggtatctctgtttctaatggttctgttgcttctttcactttggctccaggtgctgtttctgtttggcaatactctacttcagcttctgctccacaaattggttcagttgctccaaatatgggtattccaggtaacgttgttaccattgatggtaagggttttggtactacacaaggtactgttacttttggtggtgttactgctactgttaagtcttggacttccaacagaatcgaagtttacgttcctaatatggctgctggtttgacagatgttaaggttactgccggtggtgtttcttcaaacttgtactcttacaacatcttgtccggtactcaaacctctgttgtttttacagttaagtcagctccaccaactaacttgggtgataagatctatttgaccggtaacattccagaattgggtaattggtctactgatacttctggtgcagttaacaatgctcaaggtccattattggctccaaattacccagattggttctacgttttttctgttccagccggtaagaccattcaattcaagtttttcattaagagagccgacggtacaattcaatgggaaaatggttcaaaccatgttgctactactccaactggtgctactggtaacattactgttacatggcaaaactaa

(19) United States
(12) Patent Application Publication
(10) Pub. No.: US 2025/0197830 A1
(43) Pub. Date: Jun. 19, 2025

(54) N-TERMINUS ENGINEERING OF INTRACELLULAR POLYPEPTIDES EXPRESSED IN RECOMBINANT EUKARYOTIC HOST CELLS

(71) Applicant: DANSTAR FERMENT AG, Zug (CH)

(72) Inventors: Zhiqing WANG, Lebanon, NH (US); Aaron ARGYROS, Lebanon, NH (US); Michelle OESER, Lebanon, NH (US); Alexandra-Elena PANAITIU, Lebanon, NH (US); Sivakumar PATTATHIL, Lebanon, NH (US); Jan VAN EIJK, Lebanon, NH (US)

(21) Appl. No.: 18/849,224

(22) PCT Filed: Mar. 31, 2023
(86) PCT No.: PCT/IB2023/053276

§ 371 Date: Sep. 20, 2024

Related U.S. Application Data
(60) Provisional application No. 63/326,564, filed on Apr. 1, 2022.
(60) Provisional application No. 63/386,394, filed on Dec. 7, 2022.

(51) Int. Cl. C12N 9/28; A21D 8/04; C12N 1/18

(52) U.S. Cl. CPC C12N 9/2417; A21D 8/042; A21D 8/047; C12N 1/18; C12Y 302/01001

(57) ABSTRACT

Heterologous polypeptides expressed in a recombinant eukaryotic host cell may exhibit reduced stability and/or biological activity when expressed in an intracellular form. The present disclosure provides a heterologous polypeptide comprising at least one amino acid modifications which increases its stability and/or biological activity when expressed intracellularly in a recombinant eukaryotic host cell.

### CROSS-REFERENCE TO RELATED APPLICATIONS AND DOCUMENTS

This application claims priority from U.S. provisional patent applications 63/326,564 filed on Apr. 1, 2022 and 63/386,394 filed on Dec. 7, 2022, herewith incorporated in their entirety. This application also comprises a sequence listing in electronic form which is also incorporated in its entirety.

### TECHNOLOGICAL FIELD

The present disclosure concerns polypeptides which have been modified within their N-terminal region to increase their stability when expressed in an intracellular form in a recombinant eukaryotic host cell.

### BACKGROUND

Polypeptides used in commercial applications, such as enzymes, are usually obtained from the metabolism of a host cell which is capable of expressing them natively or which has been genetically engineered to express them. While the host cell can be selected or engineered to provide the polypeptides in a secreted form, it may be advantageous to provide a host cell capable of expressing them in an intracellular form. In some instances, the polypeptides are not secreted efficiently by the host cells and thus intracellular expression is preferred. In addition, the downstream processing associated with the isolation of secreted polypeptides may be costly and thus intracellular may facilitate or reduce the cost of downstream processing. However, the stability of polypeptides expressed in an intracellular form may be reduced as they are more susceptible to co- or post-transcriptional processing, such as N-terminal acetylation, which can be detrimental to their stability. There is thus a need to maintain or increase the stability and/or biological activity of polypeptides which are intended to be expressed in an intracellular form in a eukaryotic host cell.

### SUMMARY

The present disclosure provides heterologous polypeptides whose amino acid sequences has been optimized for intracellular expression in a recombinant eukaryotic host cell to maintain and/or increase its stability and/or biological activity.

In a first aspect, the present disclosure provides a recombinant eukaryotic host cell capable of expressing a heterologous mature polypeptide in an intracellular form. The amino acid sequence of the heterologous mature polypeptide has at least one amino acid modification, when compared to the amino acid sequence of a control mature polypeptide. The at least one amino acid modification is located within the N-terminal region of the amino acid sequence of the control mature polypeptide and increases the stability and/or the biological activity of the heterologous mature polypeptide compared to the control mature polypeptide expressed in an intracellular form by a control recombinant eukaryotic host cell. In an embodiment, the at least one amino acid modification increases the thermodynamic stability and/or the kinetic stability of the heterologous mature polypeptide compared to the control mature polypeptide. In another embodiment, the recombinant eukaryotic host cell and the control recombinant eukaryotic host cell are capable of performing N-terminal acetylation. In a further embodiment, the at least one amino acid modification increases the ratio of non-N-terminally acetylated to N-terminally acetylated of the heterologous mature polypeptide, when compared to the control mature polypeptide. In still another embodiment, the at least one amino acid modification is located at position 1 and/or 2 of the amino acid sequence of the control mature polypeptide. In a further embodiment, the at least one amino acid modification comprises at least one amino acid substitution. In still a further embodiment, the heterologous mature polypeptide and the control mature polypeptide have been hydrolyzed by a methionine aminopeptidase. In another embodiment, the heterologous mature polypeptide is a globular protein. In a further embodiment, the heterologous mature polypeptide is an enzyme. In still another embodiment, the at least one amino acid modification increases the specific activity of the heterologous mature polypeptide compared to the control mature polypeptide. In yet another embodiment, the enzyme is a glycoside hydrolase, such as, for example, an amylase, a cellulase, or a hemi-cellulase. In still a further embodiment, the amylase is a maltogenic alpha-amylase. In some embodiments, the amylase comprises more than one carbohydrate binding module. In some embodiments, the control mature polypeptide has the amino acid sequence of SEQ ID NO: 2 or 3, is a variant of the amino acid sequence of SEQ ID NO: 2 or 3 lacking the at least one amino acid modification and exhibiting maltogenic alpha-amylase activity or is a fragment of the amino acid sequence of SEQ ID NO: 2 or 3 comprising the N-terminal region and exhibiting maltogenic alpha-amylase activity. In such specific embodiments, the heterologous mature polypeptide can have the amino acid sequence of any one of SEQ ID NO: 4 to 9, can be a variant of the amino acid sequence of any one of SEQ ID NO: 4 to 9 having the at least one amino acid modification and exhibiting maltogenic alpha-amylase activity or can be a fragment of the amino acid sequence of any one of SEQ ID NO: 4 to 9 comprising the N-terminal region and exhibiting maltogenic alpha-amylase activity. In some embodiments, the eukaryotic host cell is a recombinant yeast host cell, such as, for example, from Saccharomyces sp. or a Komagataella sp.

According to a second aspect, the present disclosure provides a process for making the heterologous mature polypeptide described herein. The process comprises propagating, and optionally fermenting, the recombinant eukaryotic host cell described herein under conditions allowing the expression and the intracellular accumulation of the heterologous mature polypeptide. In some embodiments, the process further comprises isolating the heterologous mature polypeptide from the recombinant eukaryotic host cell.

According to a third aspect, the present disclosure provides a population of isolated heterologous mature polypeptides obtained or obtainable by the process described herein. In an embodiment, the population comprises at least one polypeptide having the amino acid sequence of any one of SEQ ID NO: 4 to 9, is a variant of the amino acid sequence of any one of SEQ ID NO: 4 to 9 having the at least one amino acid modification and exhibiting maltogenic alpha-amylase activity or is a fragment of the amino acid sequence of any one of SEQ ID NO: 4 to 9 having the N-terminal region and exhibiting maltogenic alpha-amylase activity.

According to a fourth aspect, the present disclosure provides a combination of maltogenic alpha-amylases comprising: (i) the polypeptide having amino acid sequence of any one of SEQ ID NO: 4 to 9, being a variant of the amino acid sequence of any one of SEQ ID NO: 4 to 9 having the at least one amino acid modification and exhibiting maltogenic alpha-amylase activity or being a fragment of the amino acid sequence of any one of SEQ ID NO: 4 to 9 having the at least one amino acid modification and exhibiting maltogenic alpha-amylase activity and (ii) a further maltogenic alpha-amylase.

According to a fifth aspect, the present disclosure provides a heterologous mature polypeptide obtained from a recombinant eukaryotic host cell capable of expressing the heterologous mature polypeptide in an intracellular form. The amino acid sequence of the heterologous mature polypeptide has at least one amino acid modification, when compared to the amino acid sequence of a control mature polypeptide. The at least one amino acid modification is located within the N-terminal region of the amino acid sequence of the control mature polypeptide, increases the stability and/or the biological activity of the heterologous mature polypeptide compared to the control mature polypeptide expressed in an intracellular form by a control recombinant eukaryotic host cell. In some embodiments, the at least one amino acid modification increases the thermodynamic stability and/or the kinetic stability of the heterologous mature polypeptide compared to the control mature polypeptide. In still another embodiment, the at least one amino acid modification increases the ratio of non-N-terminally acetylated to N-terminally acetylated of the heterologous mature polypeptide, when compared to the control mature polypeptide. In yet another embodiment, the at least one amino acid modification is located at position 1 and/or 2 of the amino acid sequence of the control mature polypeptide. In specific embodiments, the at least one amino acid modification comprises at least one amino acid substitution. In some embodiments, the heterologous mature polypeptide is a globular protein. In additional embodiments, the heterologous mature polypeptide is an enzyme. In yet further embodiments, the at least one amino acid modification increases the specific activity of the heterologous mature polypeptide compared to the control mature polypeptide. In yet further embodiments, the enzyme is a glycoside hydrolase, such as, for example, an amylase, a cellulase or a hemi-cellulase. In yet another embodiment, the amylase is a maltogenic alpha-amylase. In such specific embodiments, the amylase comprises more than carbohydrate binding modules. In yet another embodiment, the heterologous mature polypeptide has the amino acid sequence of any one of SEQ ID NO: 4 to 9, is a variant of the amino acid sequence of any one of SEQ ID NO: 4 to 9 having the at least one amino acid modification and exhibiting maltogenic alpha-amylase activity or is a fragment of the amino acid sequence of any one of SEQ ID NO: 4 to 9 having the N-terminal region and exhibiting maltogenic alpha-amylase activity.

According to a sixth aspect, the present disclosure provides a process for preparing a dough or a baked product prepared from the dough, the process comprising adding an effective amount of the recombinant eukaryotic host cell described herein, the population described herein, the combination described herein and/or the heterologous mature polypeptide described herein, optionally in combination with a fermenting yeast, to the dough. In an embodiment, the process further comprises, prior to, during and/or after the addition, leavening the dough. In some embodiments, the process further comprises, after the addition, baking the dough. In still some embodiments, the process further comprises storing the dough and/or the bread product. In some embodiments, the process is used to increase the softness and/or the resilience of the baked product.

According to a seventh aspect, the present disclosure provides a method for increasing the stability and/or biological activity of a control mature polypeptide intended to be expressed in an intracellular form in a recombinant eukaryotic host cell. The method comprises introducing at least one amino acid modification within the N-terminal region of the amino acid sequence of the control mature polypeptide to obtain a modified mature polypeptide exhibiting increased stability and/or biological activity when expressed by the recombinant eukaryotic host cell when compared to the control mature polypeptide. In some embodiments, the method further comprises expressing, in the intracellular form, the modified mature polypeptide in the recombinant eukaryotic host cell. In additional embodiments, the method comprises introducing the at least one amino acid modification when the ratio of non-N-terminally acetylated to N-terminally acetylated of the control mature polypeptide has been determined to be below 1.0. In further embodiments, the method further comprises determining the presence or the absence of a N-terminal acetylation of the control mature polypeptide when expressed in the intracellular form in the control recombinant eukaryotic host cell. In still another embodiment, the method is for increasing the ratio of non-N-terminally acetylated to N-terminally acetylated of the modified mature polypeptide, when compared to the control mature polypeptide. In some embodiments, the method further comprises determining the presence or the absence of a N-terminal acetylation of the modified mature polypeptide when expressed in the intracellular form in the recombinant eukaryotic host cell. In still another embodiment, the method comprises modifying (and in further embodiments substituting) an amino acid residue located at position 1 and/or 2 of the amino acid sequence of the control mature polypeptide.

### DETAILED DESCRIPTION OF THE DRAWINGS

Having thus generally described the nature of the invention, reference will now be made to the accompanying drawings, showing by way of illustration, a preferred embodiment thereof, and in which:

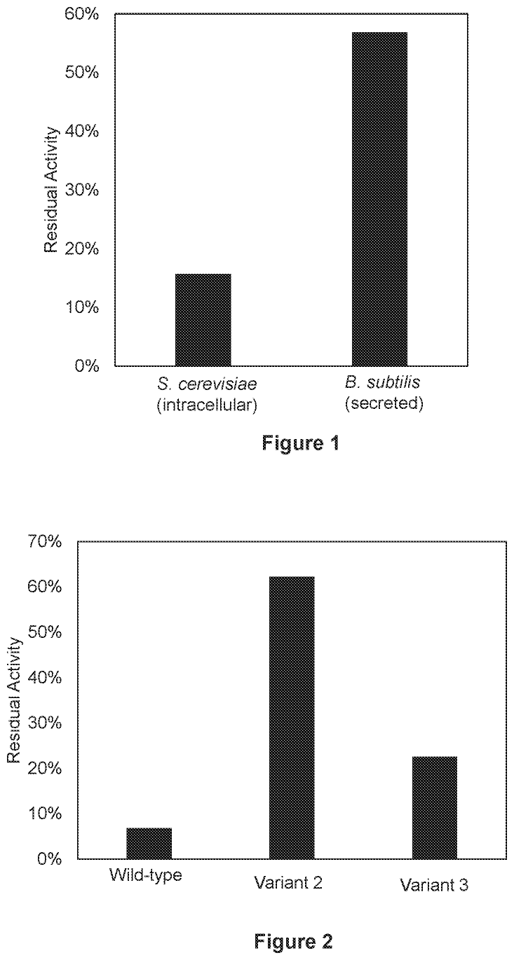

FIG. 1 compares the thermostability of a maltogenic amylase having the amino acid sequence of SEQ ID NO: 2 expressed in intracellular form by Saccharomyces cerevisiae and expressed in a secreted form by Bacillus subtilis. The residual activity of the enzyme after a heat challenge at 85° C. for 5 min (provided as a percentage of activity of the enzyme without a heat challenge) is presented in function of the host and the form expression used.

FIG. 2 compares the thermostability of the maltogenic amylase having the amino acid sequence of SEQ ID NO: 2 (wild-type) and two variants (2 and 3) all expressed in intracellular form in S. cerevisiae. The residual activity of the enzyme after a heat challenge at 85° C. for 5 min (provided as a percentage of activity of the enzyme without a heat challenge) is presented in function of the amino acid sequence of the enzyme expressed.

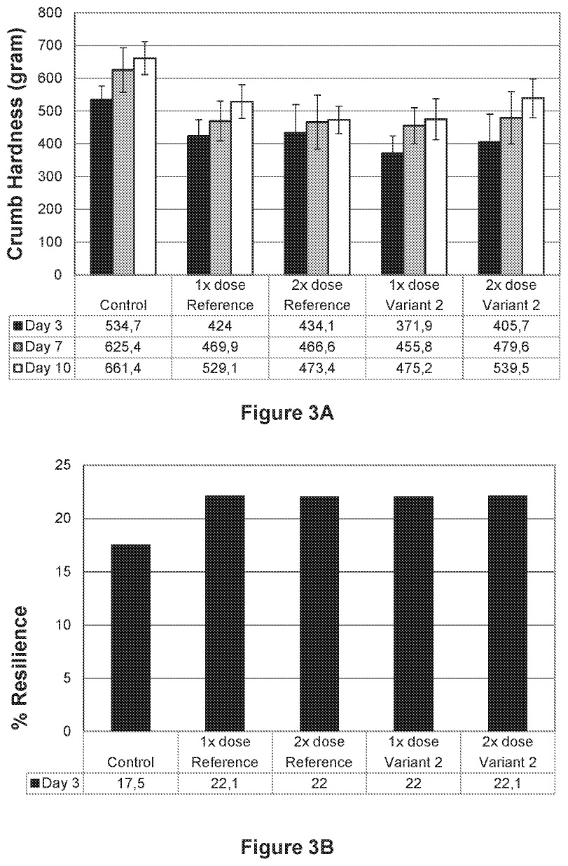

FIGS. 3A and 3B provide the staling test results of regular sandwich breads obtained in the absence of an exogenous source of maltogenic alpha-amylase (control), different doses of the wild-type enzyme (reference) or variant 2. (A) Results are shown as crumb hardness (in g) at pH 5.5 in function of the enzyme, the dose and the number of days (black bars=day 3; grey bars=day 7; white bars=day 10). (B) Results are shown as resilience (%) at pH 5.5 in function of the enzyme, the dose and the number of days (black bars=day 3).

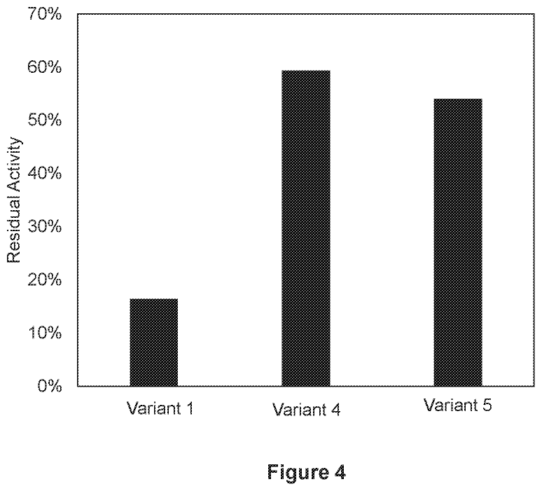

FIG. 4 compares the thermostability of the maltogenic amylase having the amino acid sequence of SEQ ID NO: 3 (e.g., Variant 1) and two variants (4 and 5) all expressed in intracellular form in S. cerevisiae. The residual activity of the enzyme after a heat challenge at 85° C. for 5 min (provided as a percentage of activity of the enzyme without a heat challenge) is presented in function of the amino acid sequence of the enzyme expressed.

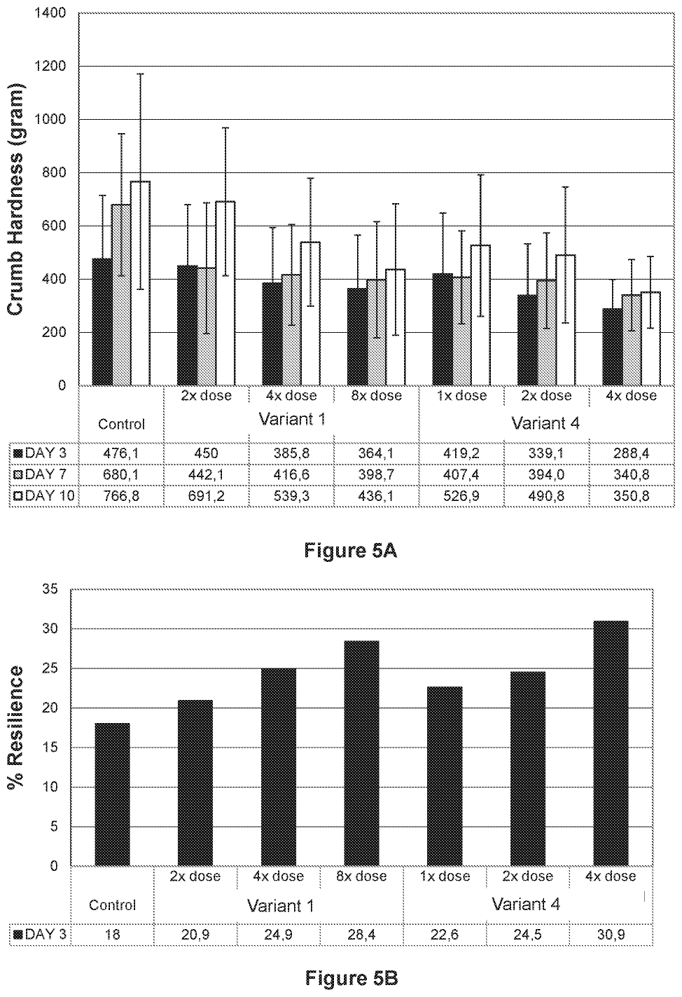

FIGS. 5A and 5B provide the staling test results of sourdough breads obtained in the absence of an exogenous source of maltogenic alpha-amylase (control), different doses of variant 1 or 4. (A) Results are shown as crumb hardness (in g) at pH 4.5 in function of the enzyme, the dose and the number of days (black bars=day 3; grey bars=day 7; white bars=day 10). (B) Results are shown as resilience (%) at pH 4.5 in function of the enzyme, the dose and the number of days (black bars=day 3; grey bars=day 7; white bars=day 10).

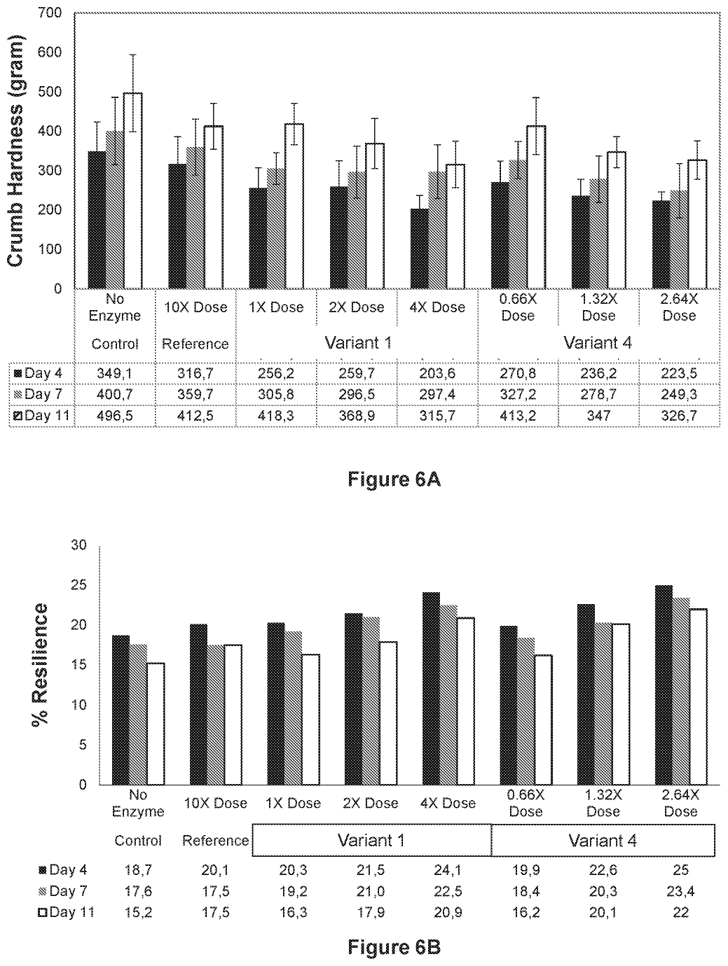

FIGS. 6A and 6B provide the staling test results of sweetened breads obtained in the absence of an exogenous source of maltogenic alpha-amylase (control), a reference maltogenic alpha-amylase (reference) or different doses of variant 1 or 4. (A) Results are shown as crumb hardness (in g) at pH 5.5 in function of the enzyme, the dose and the number of days (black bars=day 3; grey bars=day 7; white bars=day 10). (B) Results are shown as resilience (%) at pH 5.5 in function of the enzyme, the dose and the number of days (black bars=day 3; grey bars=day 7; white bars=day 10).

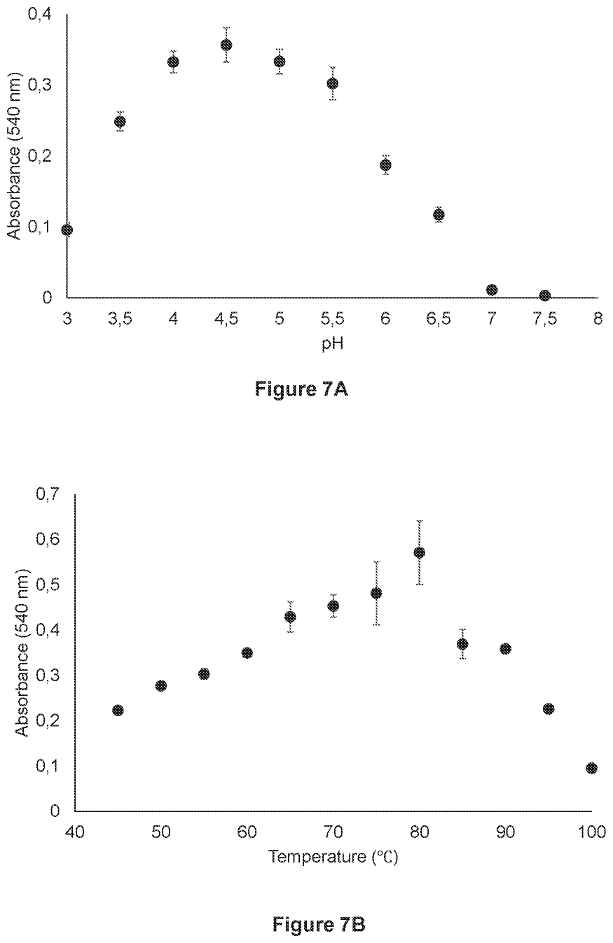

FIGS. 7A and 7B provides the activity of variant 4 (A) at different pH and (B) different temperatures. Results are shown as absorbance at 540 nm in function of the conditions tested.

### DETAILED DESCRIPTION

The present disclosure provides polypeptides (referred to as heterologous mature polypeptides) having an amino acid sequence which has been optimized to maintain and/or increase their stability and/or biological activity when expressed in an intracellular form by a recombinant eukaryotic host cell, when compared to control mature polypeptides lacking such optimization. As used in the context of the present disclosure, the expressions “in an intracellular form” and “intracellularly” refer to heterologous mature polypeptides intended to remain inside the recombinant eukaryotic host cell after they have been expressed. It is understood that polypeptides intended to be exported or secreted outside the recombinant eukaryotic host cell can transiently be found inside the recombinant eukaryotic host cell in an immature form (e.g., a form which may include a cleavable signal sequence, for example, and which may be referred to as a pre-protein). Still in the context of the present disclosure, the expression “in an intracellular form” and “intracellularly” specifically excludes polypeptides which are intended to be expressed in a mature form outside the recombinant eukaryotic host cells (either as a free form or as a tethered form) even though such polypeptides need to be present transiently inside the cell prior to export/secretion.

The present disclosure concerns the optimization of the stability of heterologous mature polypeptides (e.g., heterologous polypeptides in a mature form) when compared to control mature polypeptides (e.g., control polypeptides in a mature form). The expressions “polypeptide in a mature form” or “mature polypeptide” refer to polypeptides which have been processed by the recombinant eukaryotic host cell's peptidase(s)/protease(s) during and/or after translation. In some embodiments, peptidases/proteases can be native to the recombinant eukaryotic host cell. In further embodiments, the peptidases/proteases can be engineered to improve their biological activity. In other embodiments, the peptidases/proteases can be heterologous to the recombinant eukaryotic host cell. Peptidases involved in the maturation of polypeptides from in an immature form (e.g., uncleaved) in eukaryotes include, without limitation, signal peptidases, aminopeptidases (such as, for example, methionine aminopeptidases), and carboxypeptidases. Proteases involved in the maturation of polypeptides involved in the maturation of polypeptides from an immature form (e.g., uncleaved) in eukaryotes include, without limitations, serine proteases (for example KEX1, KEX2, and/or STE13), cysteine proteases, aspartyl proteases, threonine proteases, glutamic proteases, and metalloproteases. In some embodiments, the mature polypeptide is not cleaved by any of the recombinant eukaryotic host cell's peptidases and the amino acid sequence of its mature form corresponds to the amino acid sequence of the nascently translated polypeptide. In other embodiments, the mature polypeptide is cleaved by at least one of the recombinant eukaryotic host cell's peptidase(s) and the amino acid sequence of its mature form corresponds to a fragment of the amino acid sequence of the nascently translated polypeptide (considered, in this embodiment, the immature form of the polypeptide).

It is understood that, in the context of the present disclosure, the heterologous mature polypeptide is intended to be provided in an intracellular form and will usually lack a signal sequence. However, in some embodiments in which the immature form of the heterologous mature polypeptide includes a further localization signal (such as a nucleus localization signal, an ER localization signal or a mitochondrion localization signal), the immature form of the heterologous polypeptide can be cleaved by a signal peptidase to remove its signal sequence (in part or entirely). Therefore, the immature form of the heterologous polypeptide can be a substrate of a signal peptidase and the heterologous mature polypeptide can be a product of the signal peptidase. In other embodiments, the (immature) heterologous polypeptide lacks a signal sequence.

In other embodiments, the immature form of the heterologous polypeptide can be cleaved by an aminopeptidase to remove at least one, and in some embodiments, a plurality of N(amino)-terminal amino acid residues. Therefore, the immature form of the heterologous polypeptide can be a substrate of an aminopeptidase and the heterologous mature polypeptide can be an enzymatic product of the aminopeptidase. In yet a further embodiment, the immature form of the heterologous polypeptide can be cleaved by a methionine aminopeptidase to remove at least one N-terminal methionine residue. Therefore, the immature form of the heterologous polypeptide can be the substrate of a methionine aminopeptidase and the heterologous mature polypeptide can be an enzymatic product of the methionine aminopeptidases.

In other embodiments, the (immature) polypeptide can be cleaved by a carboxypeptidase to remove at least one, and in some embodiments, a plurality of C(carboxy)-terminal amino acid residues. Therefore, the immature form of the heterologous polypeptide can be the substrate of a carboxypeptidase and the heterologous mature polypeptide can be the enzymatic product of the carboxypeptidase.

The amino acid sequence of the heterologous mature polypeptide of the present disclosure includes at least one amino acid modification when compared to the amino acid sequence of a control mature polypeptide. The at least one amino acid modification is located within the N-terminal region of the control mature polypeptide. In some embodiments, the N-terminal region of the control mature polypeptide includes the first 50, 40, 30, 20 or 10 amino acid residues starting at the last residue at the N-terminus of the heterologous/control mature polypeptide. For example, the at least one amino acid modification can be located at positions 1-50, 1-40, 1-30, 1-20, 1-10, 1-9, 1-8, 1-7, 1-6, 1-5, 1-4, 1-3 or 1-2 of the control mature polypeptide. In embodiments, the heterologous mature polypeptide is in a globular form and the at least one amino acid modification favors the stability of the globular form. In still another embodiment, the heterologous mature polypeptide comprises physico-chemical interactions (including H bond(s) and/or salt bridge(s)) between the N-terminal and the C-terminal amino acid residues and the at least one amino acid modification stabilizes the physico-chemical interactions between the N-terminal and the C-terminal amino acid residues of the heterologous mature polypeptide (compared to the control mature polypeptide). In still another embodiment, the heterologous mature polypeptide comprises physico-chemical interactions (including H bond(s) and/or salt bridge(s)) between the ultimate N-terminal amino acid residue and the ultimate C-terminal amino acid residue and the at least one amino acid modification stabilizes the physico-chemical interactions between the ultimate N-terminal and the ultimate C-terminal amino acid residues of the heterologous mature polypeptide (when compared to the control mature polypeptide). In some further embodiments, the heterologous mature polypeptide includes, at its C-terminus, a plurality of beta-sheets which are stabilized by the presence of the at least one amino acid modification.

In some embodiments, the at least one amino acid modification can be located at position 1 of the heterologous mature/control polypeptide. In specific embodiments, the heterologous mature polypeptide includes an amino acid substitution at position 1. It will be appreciated that, in some embodiments, the mature form of the heterologous/control polypeptide can have a methionine residue at position 1 which is cleaved by a methionine aminopeptidase to generate the heterologous/control mature polypeptide. In such embodiments, the at least one amino acid modification is located at position 2 of the immature form of the heterologous/control polypeptide.

In other embodiments, the at least one amino acid modification can be located at position 2 of the heterologous mature/control polypeptide. In specific embodiments, the mature polypeptide includes an amino acid substitution at position 2 of the mature control mature polypeptide. It will be appreciated that, in some embodiments, the immature form of the heterologous/control polypeptide can have a methionine residue at position 1 which is cleaved by a methionine aminopeptidase to generate the heterologous/control mature polypeptide. In such embodiments, the at least one amino acid modification can be located at position 3 of the immature form of the heterologous/control polypeptide.

In additional embodiments, the at least one amino acid modification can be located at positions 1 and 2 of the heterologous mature/control polypeptide. In specific embodiments, the mature polypeptide includes amino acid substitutions at position 1 and 2 of the heterologous/control mature polypeptide. It will be appreciated that, in some embodiments, the immature form of the heterologous/control polypeptide can have a methionine residue at position 1 which is cleaved by a methionine aminopeptidase to generate the heterologous/control mature polypeptide. In such embodiments, the amino acid modifications can be located at positions 2 and 3 of the immature form of the heterologous/control polypeptide.

The at least one amino acid modification can include an amino acid addition, an amino acid deletion and/or an amino acid substitution. In an embodiment, the at least one amino acid modification comprises an amino acid substitution.

In an embodiment in which the amino acid residue at position 1 of the control mature polypeptide is an alanine residue, it can be substituted in the heterologous mature polypeptide by an amino acid which is different from an alanine residue, such as, for example, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is an arginine residue, it can be substituted in the heterologous mature polypeptide by an amino acid which is different from an arginine residue, such as, for example, an alanine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is an asparagine residue, it can be substituted in the heterologous mature polypeptide by an amino acid which is different from an asparagine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is an aspartic acid residue, it can be substituted in the heterologous mature polypeptide by an amino acid which is different from an aspartic acid residue, such as, for example, an alanine residue, an arginine residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a cysteine residue, it can be substituted in the heterologous mature polypeptide by an amino acid which is different from a cysteine residue, such as, for example, an alanine residue, an arginine residue, an aspartic acid residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a glutamic acid residue, it can be substituted in the heterologous mature polypeptide by an amino acid which is different from a glutamic acid residue, such as, for example, an alanine residue, an arginine residue, an aspartic acid residue, a cysteine residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a glutamine residue, it can be substituted in the heterologous mature polypeptide by an amino acid which is different from a glutamine residue, such as, for example, an alanine residue, an arginine residue, an aspartic acid residue, a cysteine residue, a glutamic acid residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a glycine residue, it can be substituted in the heterologous mature polypeptide by an amino acid which is different from a glycine residue, such as, for example, an alanine residue, an arginine residue, an aspartic acid residue, a cysteine residue, a glutamic acid residue, a glutamine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a histidine residue, it can be substituted in the heterologous mature polypeptide by an amino acid which is different from a histidine residue, such as, for example, an alanine residue, an arginine residue, an aspartic acid residue, a cysteine residue, a glutamic acid residue, a glutamine residue, a glycine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is an isoleucine residue, it can be substituted in the heterologous mature polypeptide by an amino acid which is different from an isoleucine residue, such as, for example, an alanine residue, an arginine residue, an aspartic acid residue, a cysteine residue, a glutamic acid residue, a glutamine residue, a glycine residue, a histidine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a leucine residue, it can be substituted in the heterologous mature polypeptide by an amino acid which is different from a leucine residue, such as, for example, an alanine residue, an arginine residue, an aspartic acid residue, a cysteine residue, a glutamic acid residue, a glutamine residue, a glycine residue, a histidine residue, an isoleucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a lysine residue, it can be substituted in the heterologous mature polypeptide by an amino acid which is different from a lysine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a methionine residue, it can be substituted in the heterologous mature polypeptide by an amino acid which is different from a methionine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a phenylalanine residue, it can be substituted in the heterologous mature polypeptide by an amino acid which is different from a phenylalanine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a proline residue, a serine residue, a threonine residue, a tryptophan residue, a tyrosine residue, or a valine residue. In an embodiment in which the amino acid residue at position 1 of the control mature polypeptide is a proline residue, it can be substituted in the heterologous mature polypeptide by an amino acid which is different from a proline residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a serine residue, a threonine residue, a tryptophan residue, a tyrosine residue, or a valine residue. In an embodiment in which the amino acid residue at position 1 of the control mature polypeptide is a proline residue, it can be substituted in the heterologous mature polypeptide by an amino acid which is different from a proline residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a serine residue, a threonine residue, a tryptophan residue, a tyrosine residue, or a valine residue. In an embodiment in which the amino acid residue at position 1 of the control mature polypeptide is a serine residue, it can be substituted in the heterologous mature polypeptide by an amino acid which is different from a serine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a threonine residue, a tryptophan residue, a tyrosine residue, or a valine residue. In an embodiment in which the amino acid residue at position 1 of the control mature polypeptide is a serine residue, it can be substituted in the heterologous mature polypeptide by a glycine residue. In an embodiment in which the amino acid residue at position 1 of the control mature polypeptide is a serine residue, it can be substituted in the heterologous mature polypeptide by a threonine residue. In an embodiment in which the amino acid residue at position 1 of the control mature polypeptide is a threonine residue, it can be substituted in the heterologous mature polypeptide by an amino acid which is different from a threonine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a tryptophan residue, a tyrosine residue, or a valine residue. In an embodiment in which the amino acid residue at position 1 of the control mature polypeptide is a tryptophan residue, it can be substituted in the heterologous mature polypeptide by an amino acid which is different from a tryptophan residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tyrosine residue, or a valine residue. In an embodiment in which the amino acid residue at position 1 of the control mature polypeptide is a tyrosine residue, it can be substituted in the heterologous mature polypeptide by an amino acid which is different from a tyrosine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, or a valine residue. In an embodiment in which the amino acid residue at position 1 of the control mature polypeptide is a valine residue, it can be substituted in the heterologous mature polypeptide by an amino acid which is different from a valine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, or a tyrosine residue.

In an embodiment in which the amino acid residue at position 2 of the control mature polypeptide is an alanine residue, it can be substituted in the heterologous mature polypeptide by an amino acid which is different from an alanine residue, such as, for example, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is an arginine residue, it can be substituted in the heterologous mature polypeptide by an amino acid which is different from an arginine residue, such as, for example, an alanine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is an asparagine residue, it can be substituted in the heterologous mature polypeptide by an amino acid which is different from an asparagine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is an aspartic acid residue, it can be substituted in the heterologous mature polypeptide by an amino acid which is different from an aspartic acid residue, such as, for example, an alanine residue, an arginine residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a cysteine residue, it can be substituted in the heterologous mature polypeptide by an amino acid which is different from a cysteine residue, such as, for example, an alanine residue, an arginine residue, an aspartic acid residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a glutamic acid residue, it can be substituted in the heterologous mature polypeptide by an amino acid which is different from a glutamic acid residue, such as, for example, an alanine residue, an arginine residue, an aspartic acid residue, a cysteine residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a glutamine residue, it can be substituted in the heterologous mature polypeptide by an amino acid which is different from a glutamine residue, such as, for example, an alanine residue, an arginine residue, an aspartic acid residue, a cysteine residue, a glutamic acid residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a glycine residue, it can be substituted in the heterologous mature polypeptide by an amino acid which is different from a glycine residue, such as, for example, an alanine residue, an arginine residue, an aspartic acid residue, a cysteine residue, a glutamic acid residue, a glutamine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a histidine residue, it can be substituted in the heterologous mature polypeptide by an amino acid which is different from a histidine residue, such as, for example, an alanine residue, an arginine residue, an aspartic acid residue, a cysteine residue, a glutamic acid residue, a glutamine residue, a glycine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is an isoleucine residue, it can be substituted in the heterologous mature polypeptide by an amino acid which is different from an isoleucine residue, such as, for example, an alanine residue, an arginine residue, an aspartic acid residue, a cysteine residue, a glutamic acid residue, a glutamine residue, a glycine residue, a histidine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a leucine residue, it can be substituted in the heterologous mature polypeptide by an amino acid which is different from a leucine residue, such as, for example, an alanine residue, an arginine residue, an aspartic acid residue, a cysteine residue, a glutamic acid residue, a glutamine residue, a glycine residue, a histidine residue, an isoleucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a lysine residue, it can be substituted in the heterologous mature polypeptide by an amino acid which is different from a lysine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a methionine residue, it can be substituted in the heterologous mature polypeptide by an amino acid which is different from a methionine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a phenylalanine residue, it can be substituted in the heterologous mature polypeptide by an amino acid which is different from a phenylalanine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a proline residue, a serine residue, a threonine residue, a tryptophan residue, a tyrosine residue, or a valine residue. In an embodiment in which the amino acid residue at position 2 of the control mature polypeptide is a proline residue, it can be substituted in the heterologous mature polypeptide by an amino acid which is different from a proline residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a serine residue, a threonine residue, a tryptophan residue, a tyrosine residue, or a valine residue. In an embodiment in which the amino acid residue at position 2 of the control mature polypeptide is a proline residue, it can be substituted in the heterologous mature polypeptide by an amino acid which is different from a proline residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a serine residue, a threonine residue, a tryptophan residue, a tyrosine residue, or a valine residue. In an embodiment in which the amino acid residue at position 2 of the control mature polypeptide is a serine residue, it can be substituted in the heterologous mature polypeptide by an amino acid which is different from a serine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a threonine residue, a tryptophan residue, a tyrosine residue, or a valine residue. In an embodiment in which the amino acid residue at position 2 of the control mature polypeptide is a serine residue, it can be substituted in the heterologous mature polypeptide by a glycine residue. In an embodiment in which the amino acid residue at position 2 of the control mature polypeptide is a serine residue, it can be substituted in the heterologous mature polypeptide by a threonine residue. In an embodiment in which the amino acid residue at position 2 of the control mature polypeptide is a threonine residue, it can be substituted in the heterologous mature polypeptide by an amino acid which is different from a threonine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a tryptophan residue, a tyrosine residue, or a valine residue. In an embodiment in which the amino acid residue at position 2 of the control mature polypeptide is a tryptophan residue, it can be substituted in the heterologous mature polypeptide by an amino acid which is different from a tryptophan residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tyrosine residue, or a valine residue. In an embodiment in which the amino acid residue at position 2 of the control mature polypeptide is a tyrosine residue, it can be substituted in the heterologous mature polypeptide by an amino acid which is different from a tyrosine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, or a valine residue. In an embodiment in which the amino acid residue at position 2 of the control mature polypeptide is a valine residue, it can be substituted in the heterologous mature polypeptide by an amino acid which is different from a valine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, or a tyrosine residue.

In an embodiment, the amino acid residue at position 1 of the parental/control mature polypeptide is a serine residue and the ultimate amino acid residue at the carboxyl-terminus of the parental/control polypeptide is an asparagine residue. In further embodiments, the amino acid residue at position 1 of the heterologous mature polypeptide can be a glycine or a threonine residue and the ultimate amino acid residue at the carboxyl-terminus of the parental/control polypeptide is an asparagine residue.

The presence of the at least one amino acid modification, such as the at least one amino acid substitution, causes an increase in stability and/or biological activity in the heterologous mature polypeptide, when compared to the control mature polypeptide. In embodiments, the presence of the at least one amino acid modification can cause an increase in thermodynamic stability and/or kinetic stability of the heterologous mature polypeptide, when compared to the control mature polypeptide (having been expressed in an intracellular form in the control recombinant eukaryotic host cell). As it is known in the art, the expression “thermodynamic stability” is understood to refer to the difference in Gibbs' free energy between a folded (or active) and an unfolded (or inactive) state. As it is also known in the art, the expression “kinetic stability” of the heterologous mature is understood as the rate at which a polypeptide switched between an active to an inactive state. In some embodiments, the increase in thermodynamic stability/kinetic stability can be observed as an increase in thermostability of the polypeptide which can be assessed for example, by determining the residual activity after a heat challenge and/or the melting temperature of the polypeptide. The increase in thermodynamic stability/kinetic stability can be, in embodiments in which the polypeptide is an enzyme, an increase in specific enzyme activity (e.g., the number of enzyme units/vol by the concentration of the protein in weight/vol). In some embodiments, the heterologous mature polypeptide (which can be, in some embodiments a maltogenic alpha-amylase) exhibits an increase in residual activity of at least 0.5, 0.6, 0.7, 0.8, 0.9, 1, 2, 3, 4-fold or more after a heat challenge at 85° C. for 5 min, when compared to a corresponding control polypeptide (wherein both of the heterologous/control mature polypeptides have been expressed in an intracellular form by a recombinant eukaryotic (such as a yeast) host cell). In some embodiments, the heterologous mature polypeptide (which can be, in some embodiments, a maltogenic alpha-amylase) exhibits an increase in melting temperature of at least of 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6° C. or more when measured at pH 5.5, when compared to a corresponding control mature polypeptide (when both the heterologous/control mature polypeptides have been expressed by a recombinant eukaryotic (such as a yeast) host cell in an intracellular form). In some embodiments, the heterologous mature polypeptide (which can be, in some embodiments, a maltogenic alpha-amylase) exhibits an optimal pH of at least about 3.5, 4.0, 4.5, 5.0, or 5.5. In some embodiments, the heterologous mature polypeptide (which can be, in some embodiments, a maltogenic alpha-amylase) exhibits an optimal pH of at most about 5.5, 5.0, 4.5, 4.0, or 3.5. In still another embodiment, the heterologous mature polypeptide (which can be, in some embodiments, a maltogenic alpha-amylase) exhibits an optimal pH of between about 3.5 and 5.5, and in some further embodiments, of about 4.5. In some embodiments, the heterologous mature polypeptide (which can be, in some embodiments, a maltogenic alpha-amylase) exhibits an optimal temperature of at least about 65, 70, 75, 80, or 85° C. In some embodiments, the heterologous mature polypeptide (which can be, in some embodiments, a maltogenic alpha-amylase) exhibits an optimal temperature of at most about 85, 80, 75, 70, or 65° C. In still another embodiment, the heterologous mature polypeptide (which can be, in some embodiments, a maltogenic alpha-amylase) exhibits an optimal temperature of between about 65 to 85° C., and in some further embodiments, of between about 80 to 85° C., of about 80° C.

In the context of the present disclosure, the heterologous mature polypeptide exhibits higher stability and/or biological activity than a corresponding control polypeptide which has expressed in an intracellular form in a control recombinant eukaryotic host cell. As used in the context of the present disclosure, a “control mature polypeptide” refers to a polypeptide which, when expressed in an intracellular form by a control recombinant yeast host cell, exhibits a reduction in stability and/or biological activity when compared, for example, to the heterologous mature polypeptide (having been expressed by the recombinant eukaryotic host cell). As indicated above, the control mature polypeptide lacks the at least one amino acid modification that is present in the heterologous mature polypeptide.

In some embodiments, the presence of the at least one amino acid modification, such as the at least one amino acid substitution, increases the probability of the heterologous mature polypeptide, when compared to the probability control mature polypeptide, of remaining in a non-N-terminal acetylated form. In alternative embodiments, the presence of the at least one amino acid modification, such as the at least one amino acid substitution, in a population of heterologous mature polypeptides, increases the ratio of non-N-terminally acetylated (e.g., unacetylated) heterologous mature polypeptides to N-terminal acetylated heterologous mature polypeptides. Otherwise stated, in some embodiments, the presence of the at least one amino acid modification, such as the at least one amino acid substitution, decreases the probability of the heterologous mature polypeptide, when compared to the probability control mature polypeptide, of being submitted to N-terminal acetylation. In alternative embodiments, the presence of the at least one amino acid modification, such as the at least one amino acid substitution, in a population of heterologous mature polypeptides, decreases the ratio of N-terminally acetylated heterologous mature polypeptides to non-N-terminal acetylated (e.g., unacetylated) heterologous mature polypeptides. Without being bound to theory, populations of polypeptides expressed in an intracellular form in a recombinant yeast host cells which included a higher proportion of N-terminally acetylated polypeptide exhibited lower thermostability, whereas populations of polypeptides expressed in an intracellular form in a recombinant yeast host cells which included a lower proportion of N-terminally acetylated exhibited higher thermostability. It was also noted that the by introducing an amino acid modification (such as a substitution) in the N-terminal region (such as at position 1) of the polypeptides, it was possible to reduce the ratio of N-terminally acetylated polypeptide in the population of heterologous polypeptides obtained. As such, the present disclosure provides a population of heterologous mature polypeptide obtained from the expression in a recombinant eukaryotic host cell having a ratio of non-N-terminally acetylated heterologous polypeptide to N-terminally acetylated heterologous polypeptide of at least 1:1, and in some embodiments, of at least 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1 or higher.

In some embodiments, the recombinant eukaryotic host cell of the present disclosure can be capable of or competent of N-terminal acetylation. In some embodiments, the recombinant eukaryotic host cells of the present disclosure lack any intentional genetic modification to modulate their ability to perform N-terminal acetylation. In some embodiments, the recombinant eukaryotic host cell of the present disclosure can be selected based on its reduced ability to perform N-terminal acetylation. In some embodiments, the recombinant eukaryotic host cell of the present disclosure can be genetically engineered to reduce (but not abrogate) its ability to perform N-terminal acetylation.

In some embodiments, the heterologous mature polypeptide can be derived from a parental mature polypeptide lacking the at least one amino acid modification. In some embodiments, the parental mature polypeptide is natively expressed (a native polypeptide) by a prokaryotic host cell (either in a secreted or intracellular form) or a eukaryotic host (in a secreted form). In additional embodiments, the parental mature polypeptide is a variant of the native polypeptide (lacking the at least one amino acid modification and exhibiting the biological activity of the native polypeptide). In still additional embodiments, the parental mature polypeptide is a fragment of the native polypeptide (lacking the at least one amino acid substitution, comprising the N-terminal region and exhibiting the biological activity of the native polypeptide). In still further embodiments, the parental mature polypeptide is not submitted to N-terminal acetylation when expressed by a prokaryotic host cell (either in a secreted or intracellular form) or a eukaryotic host (in a secreted form). In yet further embodiments, the parental mature polypeptide can be submitted to N-terminal acetylation when expression in an intracellular form in a recombinant eukaryotic host cell. In such embodiment, the parental mature polypeptide could correspond to the control mature polypeptide.

In an embodiment, the heterologous mature polypeptides of the present disclosures can exhibit enzymatic activity. In some embodiments, the heterologous mature polypeptides can be an hydrolase or a lytic enzyme (EC 3). In still another embodiment, the lytic enzyme can be a glycoside hydrolase or a glycosylase (E.C. 3.2). In the context of the present disclosure, the term “glycoside hydrolase” refers to an enzyme involved in carbohydrate digestion, metabolism and/or hydrolysis. Glycoside hydrolases include, without limitation, amylases (GH family 13 and/or corresponding to EC 3.2.1.1), arabinofuranosidases, asparaginases (E.C. 3.5), cellulases (E.C. 3.2.1.4), cellulolytic and amylolytic accessory enzymes, endoglucanases, esterases (E.C. 3.1), galactosidases, hemicellulases, inulinases, lactases (E.C. 3.2.1.108), levanases, pectinases, peptidases, proteases (E.C. 3.4), trehalases (E.C. 3.2.1.28), xylanases, and xylosidases. In the context of the present disclosure, the term “protease” refers to an enzyme involved in protein digestion, metabolism and/or hydrolysis. In yet another embodiment, the enzyme can be an esterase. In the context of the present disclosure, the term “esterase” refers to an enzyme involved in the hydrolysis of an ester from an acid or an alcohol, including phosphatases such as phytases. Esterases include, but are not limited to, phytases, lipases, phospholipases A1 and phospholipases A2.

Amylases can be, for example, from plant, fungal and/or bacterial origin. Amylases include, but are not limited to, alpha-amylases (EC 3.2.1.1, sometimes referred to fungal alpha-amylase), maltogenic alpha-amylases (EC 3.2.1.133), glucoamylases (EC 3.2.1.3), glucan 1,4-α-maltotetraohydrolase (EC 3.2.1.60), pullulanase (EC 3.2.1.41), iso-amylase (EC 3.2.1.68) and amylomaltase (EC 2.4.1.25). In an embodiment, the one or more amylolytic enzymes can be an alpha-amylase from Aspergillus oryzae, Saccharomycopsis fibuligera (GenBank Accession #CAA29233.1 for example), and Bacillus amyloliquefaciens (GenBank Accession #ABS72727 for example); a maltogenic alpha-amylase from Geobacillus stearothermophilus (Uniprot P19531 for example); a glucan 1,4-alpha-maltotetraohydrolase from Pseudomonas saccharophila; a pullulanase from Bacillus naganoensis; a pullulanase from Bacillus acidopullulyticus; an iso-amylase from Pseudomonas amyloderamosa; and/or an amylomaltase from Thermus thermophiles. In an embodiment, the trehalase can be from Aspergillus fumigatus (GenBank Accession #XP_748551) or Neurospora crassa (GenBank Accession #XP_960845.1).

A “cellulase” can be any enzyme involved in cellulose digestion, metabolism and/or hydrolysis, including an endoglucanase, glucosidase, cellobiohydrolase, glucanase, cellobiose phosphorylase, cellodextrin phosphorylase.

Cellulolytic and amylolytic accessory enzymes can include, for example, xylanase, xylosidase, xylan esterase, arabinofuranosidase, galactosidase, mannanase, mannosidase, xyloglucanase, endoxylanase, glucuronidase, acetylxylanesterase, arabinofuranohydrolase, swollenin, glucuronyl esterase, expansin, pectinase, and feruoyl esterase protein.

The heterologous mature polypeptide can have “hemicellulolytic activity”, an enzyme involved in hemicellulose digestion, metabolism and/or hydrolysis. The term “hemicellulase” refers to a class of enzymes that catalyze the hydrolysis of hemicellulose. Several different kinds of enzymes are known to have hemicellulolytic activity including, but not limited to, xylanases and mannanases and xylan esterases, endogxylanase, glucuronidase, acetylxyl transferease, arabinofura hydrolase, feruloyl esterase, galactanase, beta-glucanase.

The heterologous mature polypeptide can have “xylanolytic activity”, an enzyme having the is ability to hydrolyze glycosidic linkages in oligopentoses and polypentoses. The term “xylanase” is the name given to a class of enzymes which degrade the linear polysaccharide beta-1,4-xylan into xylose, thus breaking down hemicellulose, one of the major components of plant cell walls. Xylanases include those enzymes that correspond to Enzyme Commission Number 3.2.1.8. The heterologous enzyme can also be a “xylose metabolizing enzyme”, an enzyme involved in xylose digestion, metabolism and/or hydrolysis, including a xylose isomerase, xylulokinase, xylose reductase, xylose dehydrogenase, xylitol dehydrogenase, xylonate dehydratase, xylose transketolase, and a xylose transaldolase protein.

The heterologous mature polypeptide can be a “pentose sugar utilizing enzyme” involved in pentose sugar digestion, metabolism and/or hydrolysis, including xylanase, arabinase, arabinoxylanase, arabinosidase, arabinofuranosidase, arabinoxylanase, arabinosidase, and arabinofuranosidase, arabinose isomerase, ribulose-5-phosphate 4-epimerase, xylose isomerase, xylulokinase, xylose reductase, xylose dehydrogenase, xylitol dehydrogenase, xylonate dehydratase, xylose transketolase, and/or xylose transaldolase. In an embodiment, the one or more xylanase enzymes can be a xylanase from Aspergillus niger (GenBank Accession #CAA03655.1)

The heterologous enzyme can have “mannan-degradading activity”, an enzyme having the is ability to hydrolyze the terminal, non-reducing R-D-mannose residues in R-D-mannosides. Mannanases are capable of breaking down hemicellulose, one of the major components of plant cell walls.

The heterologous mature polypeptide can be a “pectinase”, an enzyme, such as pectin lyase, polygalacturonase, endopolygalacturonase (EPG), pectin methyl esterase (PME). These enzymes break down pectin, a polysaccharide substrate that is found in the cell walls of plants. The heterologous mature polypeptide can have “phytolytic activity”, an enzyme catalyzing the conversion of phytic acid into inorganic phosphorus. Phytases (EC 3.2.3) can be belong to the histidine acid phosphatases, β-propeller phytases, purple acid phosphastases or protein tyrosine phosphatase-like phytases family. In an embodiment, the one or more phytase enzymes can be a phytase from Citrobacter braakii (GenBank Accession #AY471611.1).

The heterologous mature polypeptide can have “proteolytic activity”, an enzyme involved in protein digestion, metabolism and/or hydrolysis, including serine proteases, threonine proteases, cysteine proteases, aspartate proteases (e.g., proteases having aspartic activity), glutamic acid proteases and metalloproteases. In some embodiments, the heterologous enzyme having proteolytic activity is a protease enzyme. In an embodiment, the one or more protease enzymes can be a protease from Saccharomycopsis fibuligera (GenBank Accession #P22929) or Aspergillus fumigatus (GenBank Accession #P41748).

In some embodiments, the heterologous mature polypeptides can be a lyase (EC 4). For example, the lyase can cleave carbon-carbon bonds (EC 4.1), such as decarboxylases (EC 4.1.1), aldehyde lyases (EC 4.1.2), oxo acid lyases (EC 4.1.3), and others (EC 4.1.99). In another example, the lyase can cleave carbon-oxygen bonds (EC 4.2), such as dehydratases.

In a further example, the lyase can cleave carbon-nitrogen bonds (EC 4.3), carbon-sulfur bonds (EC 4.4), carbon-halide bonds (EC 4.5), phosphorus-oxygen bonds (EC 4.6 includes lyases that cleave such as adenylyl cyclase and guanylyl cyclase), etc. (including EC 4.99, such as ferrochelatase).

In an embodiment, the parental/control/heterologous mature polypeptide is an alpha-amylase. In some embodiments, the alpha-amylase includes more than one carbohydrate binding module and can be, for example, a maltogenic alpha-amylase. Maltogenic alpha-amylase activity can be determined and optionally quantified using various assays known in the art such as those based on the measurement of reducing sugar using 3,5-dinitrosalicylic acid or 4-nitrophenol as substrates. In some embodiments, the parental/control/heterologous mature polypeptide can be derived from a maltogenic alpha-amylase from Geobacillus sp. and, in further embodiments, from Geobacillus stearothermophilus (such as, for example, Uniprot P19531 and those described in U.S. Pat. No. 6,162,628 herein incorporated in its entirety). In yet further embodiments, the parental/control mature polypeptide has the amino acid sequence of SEQ ID NO: 2, is a variant of the amino acid sequence of SEQ ID NO: 2 (lacking the at least one amino acid modification and exhibiting maltogenic alpha-amylase activity) or is a fragment of the amino acid sequence of SEQ ID NO: 2 (comprising the N-terminal region and exhibiting maltogenic alpha-amylase activity). In yet further embodiments, the parental/control mature polypeptide has the amino acid sequence of SEQ ID NO: 3, is a variant of the amino acid sequence of SEQ ID NO: 3 (lacking the at least one amino acid modification and exhibiting maltogenic alpha-amylase activity) or is a fragment of the amino acid sequence of SEQ ID NO: 3 (comprising the N-terminal region and exhibiting maltogenic alpha-amylase activity).

In some embodiments, the heterologous mature polypeptide can have the amino acid sequence of SEQ ID NO: 4. Polypeptides having the amino acid sequence of SEQ ID NO: 4 have at least one amino acid substitution at position 1 and/or 2 (when compared to the parental mature polypeptide of SEQ ID NO: 2 or 3) as well as at one further additional amino acid substitution at position 188, 261, 288, or 676. In some embodiments, the amino acid residue at position 1 is not a serine residue, and in further embodiments, can be, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a threonine residue, a tryptophan residue, a tyrosine residue, or a valine residue. In specific embodiments, the amino acid residue at position 1 can be a glycine residue (and correspond to S1G) or a threonine residue (and correspond to S1T). Still in some embodiments, the amino acid residue at position 2 is not a serine residue and can be, for example an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a threonine residue, a tryptophan residue, a tyrosine residue, or a valine residue. In specific embodiments, the amino acid residue at position 2 can be a glycine residue (and correspond to S2G) or a threonine residue (and correspond to S2T). Still in some embodiments, the amino acid residue at position 188 is not a phenylalanine residue and can be, for example an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a proline residue, a serine residue, a threonine residue, a tryptophan residue, a tyrosine residue, or a valine residue. In specific embodiments, the amino acid residue at position 188 can be a leucine residue (and correspond to F188L). Still in some embodiments, the amino acid residue at position 261 is not an aspartic acid residue and can be, for example an alanine residue, an arginine residue, an asparagine residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In specific embodiments, the amino acid residue at position 261 can be a glycine residue (and correspond to D261G). Still in some embodiments, the amino acid residue at position 288 is not a threonine residue and can be, for example an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a tryptophan residue, a tyrosine residue, or a valine residue. In specific embodiments, the amino acid residue at position 288 can be a proline residue (and correspond to T288P). In additional embodiments, the amino acid residue at position 676 is not an alanine residue and can be, for example aspartic acid residue, an arginine residue, an asparagine residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In specific embodiments, the amino acid residue at position 676 can be a valine residue (and correspond to A676V).

In some embodiments, the heterologous mature polypeptide can have the amino acid sequence of SEQ ID NO: 5, be a variant of the amino acid sequence of SEQ ID NO: 5 (comprising the at least one amino acid modification and exhibiting maltogenic alpha-amylase activity) or be a fragment of the amino acid sequence of SEQ ID NO: 5 (comprising the N-terminal region and exhibiting maltogenic alpha-amylase activity). In some embodiments, the heterologous mature polypeptide can have the amino acid sequence of SEQ ID NO: 6, be a variant of the amino acid sequence of SEQ ID NO: 6 (comprising the at least one amino acid modification and exhibiting maltogenic alpha-amylase activity) or be a fragment of the amino acid sequence of SEQ ID NO: 6 (comprising the N-terminal region and exhibiting maltogenic alpha-amylase activity). In some embodiments, the heterologous mature polypeptide can have the amino acid sequence of SEQ ID NO: 7, be a variant of the amino acid sequence of SEQ ID NO: 7 (comprising the at least one amino acid modification and exhibiting maltogenic alpha-amylase activity) or be a fragment of the amino acid sequence of SEQ ID NO: 7 (comprising the N-terminal region and exhibiting maltogenic alpha-amylase activity). In some embodiments, the heterologous mature polypeptide can have the amino acid sequence of SEQ ID NO: 8, be a variant of the amino acid sequence of SEQ ID NO: 8 (comprising the at least one amino acid modification and exhibiting maltogenic alpha-amylase activity) or be a fragment of the amino acid sequence of SEQ ID NO: 8 (comprising the N-terminal region and exhibiting maltogenic alpha-amylase activity). In additional embodiments, the heterologous mature polypeptide can be encoded by a nucleic acid molecule having the nucleic acid sequence of SEQ ID NO: 10, be a variant of the amino acid sequence of SEQ ID NO: 10, be a fragment of the amino acid sequence of SEQ ID NO: 10 or a degenerate sequence encoding the amino acid sequence of SEQ ID NO: 8, a variant thereof or a fragment thereof (comprising the N-terminal region and exhibiting maltogenic alpha-amylase activity). In some embodiments, the heterologous mature polypeptide can have the amino acid sequence of SEQ ID NO: 9, be a variant of the amino acid sequence of SEQ ID NO: 9 (comprising the at least one amino acid modification and exhibiting maltogenic alpha-amylase activity) or be a fragment of the amino acid sequence of SEQ ID NO: 9 (comprising the N-terminal region and exhibiting maltogenic alpha-amylase activity). In additional embodiments, the heterologous mature polypeptide can be encoded by a nucleic acid molecule having the nucleic acid sequence of SEQ ID NO: 11, be a variant of the amino acid sequence of SEQ ID NO: 11, be a fragment of the amino acid sequence of SEQ ID NO: 11 or a degenerate sequence encoding the amino acid sequence of SEQ ID NO: 9, a variant thereof or a fragment thereof (comprising the N-terminal region and exhibiting maltogenic alpha-amylase activity).

In some embodiments, the heterologous mature polypeptide is a combination of a first heterologous mature polypeptide having the amino acid sequence of SEQ ID NO: 7 (a variant thereof or a fragment thereof) and a second heterologous mature polypeptide having the amino acid sequence of SEQ ID NO: 9, be a variant of the amino acid sequence of SEQ ID NO: 9 (comprising the at least one amino acid modification and exhibiting maltogenic alpha-amylase activity) or be a fragment of the amino acid sequence of SEQ ID NO: 9 (comprising the N-terminal region and exhibiting maltogenic alpha-amylase activity). In additional embodiments, the heterologous mature polypeptide can be encoded by a nucleic acid molecule having the nucleic acid sequence of SEQ ID NO: 11, be a variant of the amino acid sequence of SEQ ID NO: 11, be a fragment of the amino acid sequence of SEQ ID NO: 11 or a degenerate sequence encoding the amino acid sequence of SEQ ID NO: 9, a variant thereof or a fragment thereof (comprising the N-terminal region and exhibiting maltogenic alpha-amylase activity).

As used in the context of the present disclosure, a “variant” of a polypeptide includes at least one amino acid difference when compared to the original amino acid sequence (while exhibiting the biological activity as the polypeptide having the original amino acid sequence). It is understood that the variants of the heterologous mature polypeptide include the at least one amino acid modification described herein (within the N-terminal region) as well as a further amino acid modification. It is also understood that the variants of the parental/control mature polypeptide lack the at least one acid modification described herein and include at least one further amino acid substitution. As used in the context of the present disclosure, a “fragment” includes at least one deleted amino acid residues when compared to the original amino acid sequence (while exhibiting the same biological activity as the polypeptide having the original amino acid sequence). It is understood that the fragments of the heterologous mature polypeptide include the N-terminal region comprising the at least one amino acid modification and that variants of the parental/control mature polypeptide also comprising the corresponding N-terminal region and lack the at least one acid modification. In some embodiments, the variants and the fragments can also have at least 70%, 80%, 85%, 90%, 95%, 96%, 97%, 98% or 99% identity to the original amino acid sequence. The term “percent identity”, as known in the art, is a relationship between two or more polypeptide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, NY (1988); Biocomputing: Informatics and Genome Projects (Smith, D. W., ed.) Academic Press, NY (1993); Computer Analysis of Sequence Data, Part I (Griffin, A. M., and Griffin, H. G., eds.) Humana Press, NJ (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The polypeptide variants described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide. Conservative substitutions typically include the substitution of one amino acid for another with similar characteristics, e.g., substitutions of an amino acid by another one belonging to same category determined by its side chain: within amino acids presenting hydrophobic side chain (alanine, valine, isoleucine, leucine, methionine, phenylalanine, tyrosine or tryptophan); within amino acids presenting positively charged side chain (arginine, histidine or lysine); negatively charged side chain (aspartic acid or glutamic acid) and polar-uncharged side chain (serine, threonine, asparagine or glutamine); or substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative amino acid substitutions are known in the art and are included herein. Non-conservative substitutions, such as replacing a basic amino acid with a hydrophobic one, are also well-known in the art.

A polypeptide variant can also be a conservative variant or an allelic variant. As used herein, a conservative variant refers to alterations in the amino acid sequence that do not adversely affect the biological function(s) of the polypeptide. A substitution, insertion or deletion is said to adversely affect the polypeptide when the altered sequence prevents or disrupts a biological function associated with the polypeptide. For example, the overall charge, structure or hydrophobic-hydrophilic properties of the polypeptide can be altered without adversely affecting a biological activity. Accordingly, the amino acid sequence can be altered, for example to render the peptide more hydrophobic or hydrophilic, without adversely affecting the biological activities of the polypeptide.

A polypeptide fragment can correspond to the polypeptides to which the signal peptide sequence has been removed. In additional embodiments, the polypeptide fragment can be, for example, a truncation of one or more, two or more, three or more, four, five or more, six or more, seven or more, eight or more, nine or more, ten or more, eleven or more, twelve or more amino acid residues at the carboxyl terminus of the non-truncated polypeptide or variant. Alternatively, or in combination, the fragment can be generated from removing one or more internal amino acid residues. In an embodiment, the polypeptide fragment can have at least 100, 150, 200, 250, 300, 350, 400, 450 or more consecutive amino acid residues of the original amino acid sequence or the polypeptide variant.

The heterologous mature polypeptide as well as the control mature polypeptide of the present disclosure are intended to be solely expressed inside the recombinant eukaryotic host cell, e.g., intracellularly or in an intracellular form. In some embodiments, the parental heterologous polypeptide used to generate (directly or indirectly) the control/heterologous mature polypeptide of the present disclosure can be modified to remove, if any, signal sequences present in the native amino acid sequence of the immature form of the parental polypeptide to allow for an intracellular expression. In some embodiments, the parental polypeptide of the present disclosure can be modified to replace the signal sequence with a N-terminus modification to generate the heterologous mature polypeptide.

### Recombinant Eukaryotic Host Cells Capable of Expressing the Heterologous Mature Polypeptides

The present disclosure provides recombinant eukaryotic host cells for the expression of the heterologous mature polypeptide. The present disclosure also provides control recombinant eukaryotic host cells for the expression of the control mature polypeptide. The present disclosure also provides intermediate recombinant eukaryotic host cells for the expression of the control mature polypeptide. In an embodiment, the recombinant eukaryotic host cell, the intermediate recombinant eukaryotic host cell and the control recombinant eukaryotic host cell are derived from the same genus, the same species, the same subspecies or the same strain.

In some embodiments, the recombinant eukaryotic host cell, the intermediate eukaryotic host cell and the control recombinant eukaryotic host cell can be a recombinant animal host cell, such as, for example, a recombinant mammalian host cell. In additional embodiments, the recombinant eukaryotic host cell, the intermediate eukaryotic host cell and the control recombinant eukaryotic host cell can be a recombinant plant host cell. In further embodiments, the recombinant eukaryotic host cell, the intermediate eukaryotic host cell and the control recombinant eukaryotic host cell can be a recombinant fungal host cell, such as, for example, a recombinant yeast host cell or a recombinant mold host cell. Suitable yeast host cells can be, for example, from the genus Saccharomyces, Kluyveromyces, Arxula, Debaryomyces, Candida, Komagataella, Phaffia, Schizosaccharomyces, Hansenula, Hanseniaspora, Kloeckera, Metschnikowia, Schwanniomyces, Wickerhamomyces or Yarrowia. Suitable yeast species can include, for example, S. cerevisiae, S. bulderi, S. barnetti, S. exiguus, S. uvarum, S. diastaticus, S. boulardii, K. lactis, K. marxianus or K. fragilis. In some embodiments, the yeast host cell is selected from the group consisting of Saccharomyces cerevisiae, Schizosaccharomyces pombe, Candida albicans, Komagataella phaffii, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In some additional embodiments, the yeast host cell is from Saccharomyces cerevisiae, Schizosaccharomyces pombe, Candida albicans, Komagataella phaffii, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe, Metschnikowia sinensis, Metschnikowia fructicola, Metschnikowia pulcherima, Metschnikowia zobelli, Metschnikowia shanxiensis, Wickerhamomyces anomalus, Hanseniaspora guilliermondii, Hanseniaspora pseudoguilliermondii and/or Schwanniomyces occidentalis. In some embodiments, the yeast host cell can be an oleaginous yeast cell. For example, the oleaginous yeast host cell can be from the genera Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the yeast host cell can be an oleaginous microalgae host cell (e.g., from the genera Thraustochytrium or Schizochytrium). In an embodiment, the yeast host cell is from the genus Saccharomyces and, in some embodiments, from the species Saccharomyces cerevisiae. In an embodiment, the yeast host cell is from the genus Komagataella (a genus previously known as Pichia) and, in some embodiments, from the species Komagataella phaffii (a species previously known as Pichia pastoris). In an embodiment, the recombinant yeast host cell is from the genus Torulaspora and, in some additional embodiments, from the species Torulaspora delbrueckii. Suitable fungal host cell can be, for example, from the genus Aspergillus or Trichoderma.

The recombinant eukaryotic host cell comprises one or more heterologous nucleic acid molecules encoding the mature and, in further embodiments, the immature form of the heterologous polypeptide. The control recombinant eukaryotic host cell comprises one or more heterologous nucleic acid molecules encoding the mature and, in further embodiments, the immature form of the control polypeptide. As such, the heterologous and control polypeptides as well as the nucleic acid molecules encoding same are heterologous with respect to the recombinant eukaryotic host cell expressing them. As used herein, the term “heterologous” when used in reference to a nucleic acid molecule (such as a promoter, a terminator or a coding sequence) or a polypeptide refers to a nucleic acid molecule or a polypeptide that is not natively found in the recombinant eukaryotic host cell or the control recombinant eukaryotic host cell. “Heterologous” also includes a native coding region/promoter/terminator, or portion thereof, that was introduced into the source organism in a form and/or at a location that is different from the corresponding native gene, e.g., not in its natural location in the organism's genome. The one or more heterologous nucleic acid molecule is purposively introduced into the recombinant eukaryotic host cell. For example, a heterologous nucleic acid element could be derived from a different strain of host cell, or from an organism of a different taxonomic group (e.g., different domain, kingdom, phylum, class, order, family, genus, or species, or any subgroup within one of these classifications).

The one or more heterologous nucleic acid molecule encoding the heterologous or the control polypeptide are introduced in the recombinant eukaryotic host cell or the control recombinant eukaryotic host cell to allow them to express the polypeptides. The expression of the heterologous or control polypeptide can be constitutive or induced. The expression of the heterologous polypeptide or the control polypeptide from the one or more heterologous nucleic acid molecule can occur during the propagation phase or aerobic growth of the recombinant eukaryotic host cell and/or the fermentation phase or any other anaerobic growth of the recombinant eukaryotic host cell.

In some embodiments, the one or more nucleic acid molecules encoding the heterologous and/or control mature polypeptides (as well as fragments or variants thereof) that are introduced into the recombinant eukaryotic host cells are codon-optimized with respect to the intended recipient recombinant host cell. As used herein the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules of the present disclosure comprise a coding region for the heterologous polypeptide. A DNA or RNA “coding region” is a DNA or RNA molecule which is transcribed and/or translated into a polypeptide in a cell in vitro or in vivo when placed under the control of appropriate regulatory sequences. “Suitable regulatory regions” refer to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The heterologous nucleic acid molecules described herein can comprise transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

The heterologous nucleic acid molecule can be introduced in the recombinant eukaryotic host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

In the heterologous nucleic acid molecule described herein, the promoter and the nucleic acid molecule coding for the heterologous polypeptide are operatively linked to one another. In the context of the present disclosure, the expressions “operatively linked” or “operatively associated” refers to fact that the promoter is physically associated to the nucleic acid molecule coding for the polypeptide in a manner that allows, under certain conditions, for expression of the polypeptide from the nucleic acid molecule. In an embodiment, the promoter can be located upstream (5′) of the nucleic acid sequence coding for the heterologous polypeptide. In still another embodiment, the promoter can be located downstream (3′) of the nucleic acid sequence coding for the heterologous polypeptide. In the context of the present disclosure, one or more than one promoter can be included in the nucleic acid molecule. When more than one promoter is included in the nucleic acid molecule, each of the promoters is operatively linked to the nucleic acid sequence coding for the polypeptide. The promoters can be located, in view of the nucleic acid molecule coding for the polypeptide, upstream, downstream as well as both upstream and downstream.

“Promoter” refers to a DNA fragment capable of controlling the expression of a coding sequence or functional RNA. The term “expression,” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as polypeptide binding domains (consensus sequences) responsible for the binding of the polymerase.

The promoter can be heterologous to the nucleic acid molecule encoding the heterologous polypeptide. The promoter can be heterologous or derived from a strain being from the same genus or species as the recombinant host cell. In an embodiment, the promoter is derived from the same genus, or species of the eukaryotic host cell and the polypeptide is derived from different genera from the eukaryotic host cell. One or more promoters can be used to allow the expression of the polypeptides in the recombinant yeast host cell.

In some embodiments, the host is a facultative anaerobe, such as S. cerevisiae. For facultative anaerobes, cells tend to propagate (e.g., generate biomass) or ferment (e.g., generate ethanol) depending on the availability of oxygen. Yeast cells are generally allowed to propagate before a following fermentation is conducted. In some embodiments, the promoter preferentially initiates transcription during a propagation phase such that the polypeptides are expressed during the propagation phase. As used in the context of the present disclosure, the expression “propagation phase” refers to an expansion phase of a commercial process in which the yeasts are propagated under aerobic conditions to maximize the conversion of a substrate into biomass. In some instances, the propagated biomass can be used in a following fermenting step (e.g., under anaerobic conditions) to maximize the production of one or more desired metabolites. In some additional embodiments, the propagated biomass is used directly after propagation to generate a yeast-derived composition or product enriched in heterologous mature polypeptides.

In some embodiments, the promoter or the combination of promoters present in the heterologous nucleic acid is capable of allowing the expression of the heterologous mature polypeptide and/or the control mature polypeptide during the propagation phase of the recombinant eukaryotic host cell. This will allow the accumulation of the polypeptide associated with the recombinant eukaryotic host cell prior to any subsequent use, for example fermentation. In some embodiments, the promoter allows the expression of the polypeptide during the propagation phase.

In other embodiments, the promoter or the combination of promoters present in the heterologous nucleic acid is capable of allowing the expression of the recombinant heterologous polypeptide during the anaerobic growth or culture (for example, in the fermentation phase) of the recombinant eukaryotic host cell.

The promoters that can be included in the heterologous nucleic acid molecule can be constitutive or inducible promoters. Inducible promoters include, but are not limited to glucose-regulated promoters (e.g., the promoter of the hxt7 gene (referred to as hxt7p), a functional variant or a functional fragment thereof; the promoter of the ctt1 gene (referred to as ctt1p), a functional variant or a functional fragment thereof; the promoter of the glo1 gene (referred to as glo1p), a functional variant or a functional fragment thereof; the promoter of the ygp1 gene (referred to as ygp1p), a functional variant or a functional fragment thereof; the promoter of the gsy2 gene (referred to as gsy2p), a functional variant or a functional fragment thereof), molasses-regulated promoters (e.g., the promoter of the mol1 gene (referred to as mol1p), a functional variant or a functional fragment thereof), heat shock-regulated promoters (e.g., the promoter of the glo1 gene (referred to as glo1p), a functional variant or a functional fragment thereof; the promoter of the sti1 gene (referred to as sti1p), a functional variant or a functional fragment thereof; the promoter of the ygp1 gene (referred to as ygp1p), a functional variant or a functional fragment thereof; the promoter of the gsy2 gene (referred to as gsy2p), a functional variant or a functional fragment thereof), oxidative stress response promoters (e.g., the promoter of the cup1 gene (referred to as cup1p), a functional variant or a functional fragment thereof; the promoter of the ctt1 gene (referred to as ctt1p), a functional variant or a functional fragment thereof; the promoter of the trx2 gene (referred to as trx2p), a functional variant or a functional fragment thereof; the promoter of the gpd1 gene (referred to as gpd1p), a functional variant or a functional fragment thereof; the promoter of the hsp12 gene (referred to as hsp12p), a functional variant or a functional fragment thereof), osmotic stress response promoters (e.g., the promoter of the ctt1 gene (referred to as ctt1p), a functional variant or a functional fragment thereof; the promoter of the glo1 gene (referred to as glo1p), a functional variant or a functional fragment thereof; the promoter of the gpd1 gene (referred to as gpd1p), a functional variant or a functional fragment thereof; the promoter of the ygp1 gene (referred to as ygp1p), a functional variant or a functional fragment thereof), nitrogen-regulated promoters (e.g., the promoter of the ygp1 gene (referred to as ygp1p), a functional variant or a functional fragment thereof), promoter of the adh1 gene (referred to as adh1p), a functional variant or a functional fragment thereof and methanol-inducible promoters (e.g., the promoter from the aox1 gene (referred to as aox1p), a functional variant or a functional fragment thereof). Promoters that can be included in the heterologous nucleic acid molecule of the present disclosure include, without limitation, the promoter of the tdh1 gene (referred to as tdh1p, a functional variant or a functional fragment thereof), of the hor7 gene (referred to as hor7p, a functional variant or a functional fragment thereof), of the hsp150 gene (referred to as hsp150p, a functional variant or a functional fragment thereof), of the hxt7 gene (referred to as hxt7p, a functional variant or a functional fragment thereof), of the gpm1 gene (referred to as gpm1p, a functional variant or a functional fragment thereof), of the pgk1 gene (referred to as pgk1p, a functional variant or a functional fragment thereof), of the stl1 gene (referred to as stl1p, a functional variant or a functional fragment thereof) and/or of the tef2 gene (referred to as tef2p, a functional variant or a functional fragment thereof).

In the context of the present disclosure, the expression “functional fragment of a promoter” refers to a shorter nucleic acid sequence than the native promoter which retain the ability to control the expression of the nucleic acid sequence encoding the heterologous/control mature polypeptide. Usually, functional fragments are either 5′ and/or 3′ truncation of one or more nucleic acid residue from the native promoter nucleic acid sequence. In the context of the present disclosure, the expression “functional variant of a promoter” refers to a nucleic acid sequence which differs in at least one position and still retain the ability to control the expression of the nucleic acid sequence encoding the heterologous/control mature polypeptide.

In some embodiments, the heterologous nucleic acid molecules include one or a combination of terminator sequence(s) to end the translation of the heterologous/control mature polypeptide. The terminator can be native or heterologous to the nucleic acid sequence encoding the heterologous polypeptide. In some embodiments, one or more terminators can be used. In some embodiments, the terminator comprises the terminator derived from is from the dit1 gene (dit1t, a functional variant or a functional fragment thereof), from the idp1 gene (idp1t, a functional variant or a functional fragment thereof), from the gpm1 gene (gpm1t, a functional variant or a functional fragment thereof), from the pma1 gene (pam1t, a functional variant or a functional fragment thereof), from the tdh3 gene (tdh3t, a functional variant or a functional fragment thereof), from the hxt2 gene (a functional variant or a functional fragment thereof), from the adh3 gene (adh3t, a functional variant or a functional fragment thereof), and/or from the ira2 gene (ira2t, a functional variant or a functional fragment thereof). In an embodiment, the terminator comprises or is derived from the dit1 gene (dit1t, a functional variant or a functional fragment thereof). In another embodiment, the terminator comprises or is derived adh3t and/or idp1t.

In the context of the present disclosure, the expression “functional variant of a terminator” refers to a nucleic acid sequence that has been substituted in at least one nucleic acid position when compared to the native terminator which retain the ability to end the expression of the nucleic acid sequence coding for the heterologous/control mature polypeptide. In the context of the present disclosure, the expression “functional fragment of a terminator” refers to a shorter nucleic acid sequence than the native terminator which retain the ability to end the expression of the nucleic acid sequence coding for the heterologous/control mature polypeptide.

### Process for Making the Heterologous Mature Polypeptide as Well as Compositions Comprising Same

The present disclosure provides processes for the heterologous mature polypeptide as well as compositions comprising same. In the compositions of the present disclosure, the heterologous mature polypeptides exhibit their biological activity(ies), e.g., they are considered functional. In such process, recombinant eukaryotic host cells comprising the one or more heterologous nucleic acid molecule encoding (directly or indirectly) the mature form of the heterologous polypeptides can be cultured or propagated to allow the expression and the accumulation of the heterologous mature polypeptides inside the recombinant eukaryotic host cells. The propagation step is usually conducted in a culture medium allowing the cell growth and division of the recombinant eukaryotic host cells under conditions (agitation, temperature, oxygen concentration, etc.) to favor the intracellular expression and accumulation, and optionally the maturation, of the heterologous mature polypeptides. Once the recombinant eukaryotic host cells have been propagated, they can optionally be submitted to an anaerobic growth phase (such as a fermentation step) or directly be submitted to a process for making compositions comprising the heterologous mature polypeptides. The processes of the present disclosure also include formulating the propagated and optionally fermented recombinant eukaryotic host cells into a composition comprising the heterologous mature polypeptide.

In some embodiments, the process includes a step of isolating the heterologous mature polypeptides, e.g., separating one or more components of the propagated recombinant eukaryotic host cell from the heterologous mature polypeptides to obtain an isolated fraction enriched in heterologous mature polypeptide. In some embodiments, the propagated recombinant eukaryotic host cells can be submitted to a lysis step to provide a lysed fraction comprising the heterologous mature polypeptides. The lysis step can be achieved, for example, by autolysis, a heat treatment, a pH treatment, a salt treatment, a homogenization step, as well as combinations thereof. In some embodiments, the lysed fraction can be submitted to a separating step, such as, for example, centrifugation and/or filtration, to obtain a separated fraction comprising the heterologous mature polypeptides. In some embodiments, the isolated fraction, the lysed fraction and/or the separated fraction can be submitted to a washing step to obtain a washed fraction comprising the heterologous mature polypeptide. In some embodiments, the isolated fraction, the lysed fraction, the separated fraction and/or the washed fraction can be submitted to a drying step to obtain a dried fraction comprising the heterologous mature polypeptides. In some embodiments, the dried fraction can be obtained using roller-drying, electrospray-drying, freeze-drying, spray-drying, lyophilization and/or fluid-bed drying. The process can include, in some embodiments, determining the purity, activity and/or N-terminal acetylation status of the heterologous mature polypeptides in one or more of the various fractions that can be obtained.

Alternatively, the heterologous mature polypeptides are not separated from the propagated recombinant eukaryotic host cells. In such embodiments, the heterologous mature polypeptides can be provided in living propagated recombinant eukaryotic host cells (e.g., an active preparation). The living propagated recombinant eukaryotic host cell can be provided in a liquid or semi-liquid form or in a dried form. When the recombinant eukaryotic host cells are yeast host cells, after propagation, they can be provided as a yeast cream or in a dried form (e.g., active dried yeasts). In other embodiments, the propagated recombinant eukaryotic host cells can be inactivated, totally or in part, to provide an inactivated or semi-inactive preparation. The inactivation step can be achieved, for example, by autolysis, a heat treatment, a pH treatment, a salt treatment, a homogenization step, etc.

In some embodiments, the process can include, determining the activity and/or N-terminal acetylation status of the heterologous mature polypeptides present in the living propagated eukaryotic host cells, the semi-inactive preparation or the inactive preparation.

The various compositions (including fractions and preparations) comprising the heterologous mature polypeptides can be provided in a liquid, semi-liquid or dry form. The composition can be, for example, an animal composition (e.g., a composition made from a recombinant animal host cell having expressed the heterologous mature polypeptide). The composition can be, for example, a plant composition (e.g., a composition made from a recombinant plant host cell having expressed the heterologous mature polypeptide). The composition can be, for example, a yeast composition (e.g., a composition made from a recombinant yeast host cell having expressed the heterologous mature polypeptide). The composition can be a fungal composition (e.g., a composition made from a recombinant fungal host cell having expressed the heterologous mature polypeptide).

The process can also be used to make a yeast product (e.g., a composition derived from a recombinant yeast host cell having expressed the heterologous mature polypeptide). When the yeast product is an inactivated yeast product, the process for making the yeast product broadly comprises two steps: a first step of providing propagated recombinant yeast host cells and a second step of lysing the propagated yeast host cells for making the yeast product. The process for making the yeast product can include an optional separating step, an optional washing step and/or an optional drying step. In some embodiments, the propagated recombinant yeast host cells are propagated on molasses. Alternatively, the propagated recombinant yeast host cells are propagated on a medium comprising a yeast extract.

In some embodiments, the recombinant yeast host cells can be lysed using autolysis (which can optionally be performed in the presence of additional exogenous enzymes). For example, the propagated recombinant yeast host cells may be subject to a combined heat and pH treatment for a specific amount of time (e.g., 6, 12, 18, 24, 36, 48 h or more) in order to cause the autolysis of the propagated recombinant yeast host cells to provide the lysed recombinant yeast host cells. For example, the propagated recombinant yeast host cells can be submitted to a temperature of between about 40° C. to about 70° C. or between about 50° C. to about 60° C. The propagated recombinant yeast host cells can be submitted to a temperature of at least about 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C. or 70° C. Alternatively or in combination the propagated recombinant yeast host cells can be submitted to a temperature of no more than about 70° C., 69° C., 68° C., 67° C., 66° C., 65° C., 64° C., 63° C., 62° C., 61° C., 60° C., 59° C., 58° C., 57° C., 56° C., 55° C., 54° C., 53° C., 52° C., 51° C., 50° C., 49° C., 48° C., 47° C., 46° C., 45° C., 44° C., 43° C., 42° C., 41° C. or 40° C. In another example, the propagated recombinant yeast host cells can be submitted to a pH between about 4.0 and 8.5, between about 5.0 and 7.5, or between about 5.0 and 6.0. The propagated recombinant yeast host cells can be submitted to a pH of at least about, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4 or 8.5. Alternatively or in combination, the propagated recombinant yeast host cells can be submitted to a pH of no more than 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3., 5.2, 5.1, 5.0, 4.9, 4.8, 4.7, 4.6 or 4.5. If necessary, the lysed recombinant yeast host cell can be submitted to a centrifugation and/or a filtration step to purify, at least in part, the heterologous polypeptide.

In some embodiments, the recombinant yeast host cells can be homogenized (for example using a bead-milling technique, a bead-beating or a high-pressure homogenization technique) and, as such, the process for making the yeast product can comprise a homogenizing step of the propagated recombinant yeast host cells having expressed the heterologous mature polypeptide.

The process for making the yeast product can also include a drying step. The drying step can include, for example, roller-drying, electrospray-drying, freeze-drying, spray-drying, lyophilization and/or fluid-bed drying. When the yeast product is an autolysate, the process may include directly drying the lysed recombinant yeast host cells after the lysis step without performing an additional separation of the lysed faction.

The process for making the yeast product can include a heating step. In such embodiment, care should be taken to perform the heating step so as to preserve the residual enzymatic activity of the heterologous mature polypeptides of the present disclosure.

To provide additional yeast products, it may be necessary to further separate the components of the lysed recombinant yeast host cells. For example, the cellular wall components (referred to as a “insoluble fraction”) of the lysed recombinant yeast host cell may be separated from the other components (referred to as a “soluble fraction”) of the lysed recombinant yeast host cells. This separating step can be done, for example, by using centrifugation and/or filtration. The process of the present disclosure can include one or more washing step(s) to provide the cell walls or the yeast extract. The yeast extract can be made by drying the soluble fraction obtained.

In an embodiment of the process, the soluble fraction can be further separated prior to drying. For example, the components of the soluble fraction having a molecular weight of more than 10 kDa can be separated out of the soluble fraction. This separation can be achieved, for example, by using filtration (and more specifically ultrafiltration). When filtration is used to separate the components, it is possible to filter out (e.g., remove) the components having a molecular weight less than about 10 kDa and retain the components having a molecular weight of more than about 10 kDa. The components of the soluble fraction having a molecular weight of more than 10 kDa can then optionally be dried to provide a retentate as the yeast product. When the yeast composition is an active/semi-active product, it can be submitting to a concentrating step, e.g., a step of removing part of the propagation/fermentation medium from the propagated recombinant yeast host cells. The concentrating step can include resuspending the concentrated and propagated/fermented recombinant yeast host cells in the propagation medium (e.g., unwashed preparation) or a fresh medium or aqueous solution (e.g., washed preparation).

Inactivated yeast products include, but are not limited to a yeast extract. Active/semi-active yeast products include, but are not limited to, a cream yeast, an instant dried yeast or an active-dried yeast. Inactivated fungal products, include but are not limited to a fungal extract. An active/semi-active fungal products include, but are not limited to, fungal concentrates.

In some embodiments, the heterologous mature polypeptides of the present disclosure can be in a semi-purified or a substantially purified form. As used in the context of the present disclosure, the expression “semi-purified form” refers to the fact that the heterologous mature polypeptides have been physically dissociated, at least in part, from the components of the recombinant eukaryotic host cell having expressed same. The expression “substantially purified form” refers to the fact that the heterologous mature polypeptides have been physically dissociated from the majority of the components of the recombinant eukaryotic host cells having expressed same. In an embodiment, a composition comprising the heterologous polypeptides in substantially purified form is at least 90%, 95%, 96%, 97%, 98% or 99% pure. In some embodiments, the composition comprising the heterologous mature polypeptide lacks a detectable amount of deoxyribonucleic acids from the recombinant eukaryotic host cell used to express it.

The compositions of the present disclosure can include, besides the polypeptides, variants, or fragments, a recombinant eukaryotic host cell (living, inactivated or dead) or at least one component of a recombinant eukaryotic host cell. The “at least one component of a recombinant eukaryotic host cell” can be an intracellular component and/or a component associated with the eukaryotic host cell's wall or membrane. The “at least one component of a recombinant eukaryotic host cell” can include a protein, a peptide or an amino acid, a carbohydrate and/or a lipid. The “at least one component of a recombinant eukaryotic host cell” can include a eukaryotic host cell's organelle.

The present disclosure also provides a population of heterologous mature polypeptides which have been obtained from recombinant expression, in an intracellular form, in the recombinant eukaryotic host cell. In an embodiment, the population comprises one or more heterologous mature polypeptide in a non-N-terminally acetylated form, optionally in combination with a heterologous mature polypeptide in a N-terminally acetylated form. In embodiments in which the recombinant eukaryotic host cell is capable of performing N-terminal acetylation, the population of heterologous mature polypeptides comprises at least 50%, 55%, 60%, 65%, 70%, 75%, 80%, 85%, 90%, 91%, 92%, 93%, 94% 95%, 96%, 97%, 98%, 99% or more of non-N-terminally acetylated heterologous mature polypeptides (when compared to the total heterologous mature polypeptides having been expressed by the recombinant eukaryotic host cell). In embodiments in which the recombinant eukaryotic host cell is capable of performing N-terminal acetylation, the population of heterologous mature polypeptides comprises no more than 50%, 45%, 40%, 35%, 30%, 25%, 20%, 15%, 10%, 9%, 8%, 7%, 6%, 5%, 4%, 3%, 2%, 1% or less of N-terminally acetylated heterologous mature polypeptides (when compared to the total heterologous mature polypeptides having been expressed by the recombinant eukaryotic host cell). In embodiments in which the recombinant eukaryotic host cell is capable of performing N-terminal acetylation, the population of heterologous mature polypeptides comprises a ratio of at least 1:1, and in some embodiments, of at least 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1 or higher of non-N-terminally acetylated to N-terminally acetylated heterologous mature polypeptides. In embodiments in which the recombinant eukaryotic host cell is capable of performing N-terminal acetylation, the population of heterologous mature polypeptides comprises a ratio of no more than 1:1, and in some embodiments, of at least 1:1.5, 2, 1:2.5, 1:3, 1:3.5, 1:4, 1:4.5, 1:5, 1:5.5, 1:6, 1:6.5, 1:7, 1:7.5. 1:8, 1:8.5, 1:9, 1:9.5 or lower of N-terminally acetylated to non-N-terminally acetylated heterologous mature polypeptides.

In some embodiments, the process of the present disclosure can include admixing the heterologous mature polypeptide (which can be provided in the form of a population of heterologous mature polypeptides) with a further component such as, another polypeptide or enzyme to provide a combination. When the heterologous mature polypeptide is an enzyme having maltogenic alpha-amylase activity, the heterologous mature polypeptides may be admixed with other enzymes having maltogenic alpha-amylase activity to provide a combination or blend of enzymes having maltogenic alpha-amylase activity.

### Process for Using the Heterologous Mature Polypeptide as Well as Compositions Comprising Same

In embodiments in which the heterologous mature polypeptide (a composition comprising same) exhibits maltogenic alpha-amylase activity, the heterologous mature polypeptide can be used for preparing a dough or a baked product from the dough (such as, for example, a bread). In such embodiments, the heterologous mature polypeptide can be used to reduce the retrogradation of the starch, to reduce the staling of the baked product, to reduce the crumb hardness of the baked product, to increase the percent resilience of the baked product and/or to prolong the shelf-life of the baked product (when compared, for example to a baked product prepared in the absence of the heterologous mature polypeptide).

Broadly, the process comprises contacting the heterologous mature polypeptide with the dough or part of the dough. If necessary, a fermenting yeast could be added to the dough or part of the dough. In some embodiments, a chemical leavening agent can be added to the dough or part of the dough. The process can include, in some embodiments, pre-fermenting part of the dough to obtain a preferment. The heterologous mature polypeptide (which may be provided in the preferment) can be contacted with the dough prior to and/or after the pre-fermenting step. The process can include leavening the dough (using a yeast, a chemical leavening agent or a combination of both). The heterologous mature polypeptide can be contacted with the dough prior to and/or after the leavening step. The process can include baking the dough. Alternatively, or in combination, the process can include frying the dough. It is understood that, in the context of the present disclosure, the baking/frying step(s) is(are) performed at least in part, in the presence of the heterologous mature polypeptide in the dough. The process can also include a step of storing the baked product prior to its consumption.

In an embodiment, the heterologous mature polypeptide can be provided as an intracellular component of a recombinant eukaryotic host cell which is contacted with the dough or part of the dough. In another embodiment, the heterologous mature polypeptide can be provided as an intracellular component of a recombinant yeast host cell which is contacted with the dough or part of the dough. In such embodiment, it may not be necessary to include additional exogenous maltogenic alpha-amylase and/or a fermenting yeast to the dough or part of the dough as the recombinant yeast host cell can provide both maltogenic alpha-amylase activity as well as fermentation capacity.

The heterologous mature polypeptide can be added to different types of doughs such as, for example, a leavened dough, a sponge dough, a straight dough, an unleavened dough regular dough, a non-laminated dough, a doughnut (donut) dough, an acid dough and/or a pâte sucrée (sweetened dough). Doughs include, without limitation, bread dough, cake dough, brioche dough, challah dough, crépe dough, focaccia dough, pasta dough, pizza dough, rolled-in dough, a rich dough, a pie dough, a pâte brisée, sablee dough, puff pastry dough, phyllo dough, choux pastry dough, croissant dough, kourou dough, and sourdough. The process can be used to generate various baked product, including, but not limited to, a leavened baked product, a sponge baked product, a straight baked product, an unleavened baked product, a non-laminated baked product, a bread, a brioche, challah, a crépe, a focaccia, a pasta, a pizza, rolled-in baked product, a pie, a pâte brisée, sablee, a pastry (including a puff pastry), a choux pastry, a croissant, a kourou dough, and a sourdough. The process can be used to generate various fried products, including, but not limited to, doughnuts (donuts).

In an embodiment, the heterologous mature polypeptide can be added to a dough comprising a sweetening agent, such as, for example, a dough comprising glucose, high-fructose syrup, sucrose, fructose, trehalose, molasses, honey, maple syrup, stevia or a synthetic sweetener (like sucralose for example). In specific embodiments, the heterologous mature polypeptide can be added to a dough comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30% (in baker's percentage, e.g., weight/weight of flour) of the sweetening agent. In another embodiment, the heterologous mature polypeptide can be added to a dough which has not been supplemented with an exogenous source of a sweetening agent. In another embodiment, the heterologous mature polypeptide can be added to a dough intended to be used in a product having zero net carbohydrate, e.g., having less than 0.5 g of carbohydrate per serving.

In an embodiment, the heterologous mature polypeptide can be added to an acidified dough. In specific embodiments, the heterologous mature polypeptide can be added to a dough having a pH equal to or below 5.0, 4.9, 4.8, 4.7, 4.6, 4.5, 4.4, 4.3, 4.2, 4.1 or lower.

In an embodiment, the heterologous mature polypeptide can be added to a dough comprising a lipid (a fat or an oil). In an embodiment, the oil is a vegetable oil, such as, for example a canola oil and/or a soybean oil. In a further embodiment, the heterologous mature polypeptide can be added to a dough which comprises comprising at least 1, 2, 3, 4, 5, 6, 7, 8, 9, 10, 11, 12, 13, 14, 15% (in baker's percentage, e.g., weight/weight of flour) of the lipid. In another embodiment, the heterologous mature polypeptide can be added to a dough which has not been supplemented with an exogenous source of a lipid.

Method for Increasing the Stability and/or the Biological Activity of a Polypeptide

The present disclosure provides a method for increasing the stability and/or the biological activity of a (control) mature polypeptide which is intended to be expressed in an intracellular form by a recombinant eukaryotic host cell. Broadly, the method comprises introducing at least one amino acid modification within the N-terminal region of the control mature polypeptide intended to be expressed in an intracellular form by a recombinant eukaryotic host cell to obtain a modified mature polypeptide exhibiting an increase its stability and/or biological activity.

In some embodiments, the N-terminal region of the control/modified mature polypeptide includes the first 50, 40, 30, 20 or 10 amino acid residues starting at the N-terminus of the heterologous control mature polypeptide. For example, the at least one amino acid modification can be located at positions 1-50, 1-40, 1-30, 1-20, 1-10, 1-9, 1-8, 1-7, 1-6, 1-5, 1-4, 1-3, 1-2 of the control mature polypeptide. In embodiments, the modified mature polypeptide is in a globular form and the at least one amino acid modification favors the stability of the globular form in the modified heterologous polypeptide. In still another embodiment, the modified mature polypeptide comprises physico-chemical interactions (including H bond(s) and/or salt bridge(s)) between the N-terminal and the C-terminal amino acid residues and the at least one amino acid modification stabilizes the physico-chemical interactions between the N-terminal and the C-terminal amino acid residues of the modified mature polypeptide (compared to the control mature polypeptide). In still another embodiment, the modified mature polypeptide comprises physico-chemical interactions (including H bond(s) and/or salt bridge(s)) between the ultimate N-terminal amino acid residue and the ultimate C-terminal amino acid residue and the at least one amino acid modification stabilizes the physico-chemical interactions between the ultimate N-terminal and the ultimate C-terminal amino acid residues of the modified mature polypeptide (when compared to the control mature polypeptide). In some further embodiments, the modified mature polypeptide includes, at its C-terminus, a plurality of beta-sheets which are stabilized by the presence of the at least one amino acid modification.

In some embodiments, the at least one amino acid modification can be located at position 1 of the modified/control polypeptide. In specific embodiments, the modified mature polypeptide includes an amino acid substitution at position 1. It will be appreciated that, in some embodiments, the immature form of the modified/control polypeptide can have a methionine residue at position 1 which is cleaved by a methionine aminopeptidase to generate the modified/control mature polypeptide. In such embodiments, the at least one amino acid modification can be located at position 2 of the immature form of the modified/control polypeptide.

In other embodiments, the at least one amino acid modification can be located at position 2 of the modified mature/control polypeptide. In specific embodiments, the mature polypeptide includes an amino acid substitution at position 2 of the mature control mature polypeptide. It will be appreciated that, in some embodiments, the immature form of the modified/control polypeptide can have a methionine residue at position 1 which is cleaved by a methionine aminopeptidase to generate the modified/control mature polypeptide. In such embodiments, the at least one amino acid modification can be located at position 3 of the immature form of the modified/control polypeptide.

In additional embodiments, the at least one amino acid modification can be located at positions 1 and 2 of the modified mature/control polypeptide. In specific embodiments, the mature polypeptide includes amino acid substitutions at position 1 and 2 of the modified/control mature polypeptide. It will be appreciated that, in some embodiments, the immature form of the modified/control polypeptide can have a methionine residue at position 1 which is cleaved by a methionine aminopeptidase to generate the modified/control mature polypeptide. In such embodiments, the amino acid modifications can be located at positions 2 and 3 of the immature form of the modified/control polypeptide.

The at least one amino acid modification can include an amino acid addition, an amino acid deletion and/or an amino acid substitution. In an embodiment, the at least one amino acid modification comprises an amino acid substitution.

In an embodiment in which the amino acid residue at position 1 of the control mature polypeptide is an alanine residue, it can be substituted in the modified mature polypeptide by an amino acid which is different from an alanine residue, such as, for example, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is an arginine residue, it can be substituted in the modified mature polypeptide by an amino acid which is different from an arginine residue, such as, for example, an alanine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is an asparagine residue, it can be substituted in the modified mature polypeptide by an amino acid which is different from an asparagine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is an aspartic acid residue, it can be substituted in the modified mature polypeptide by an amino acid which is different from an aspartic acid residue, such as, for example, an alanine residue, an arginine residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a cysteine residue, it can be substituted in the modified mature polypeptide by an amino acid which is different from a cysteine residue, such as, for example, an alanine residue, an arginine residue, an aspartic acid residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a glutamic acid residue, it can be substituted in the modified mature polypeptide by an amino acid which is different from a glutamic acid residue, such as, for example, an alanine residue, an arginine residue, an aspartic acid residue, a cysteine residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a glutamine residue, it can be substituted in the modified mature polypeptide by an amino acid which is different from a glutamine residue, such as, for example, an alanine residue, an arginine residue, an aspartic acid residue, a cysteine residue, a glutamic acid residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a glycine residue, it can be substituted in the modified mature polypeptide by an amino acid which is different from a glycine residue, such as, for example, an alanine residue, an arginine residue, an aspartic acid residue, a cysteine residue, a glutamic acid residue, a glutamine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a histidine residue, it can be substituted in the modified mature polypeptide by an amino acid which is different from a histidine residue, such as, for example, an alanine residue, an arginine residue, an aspartic acid residue, a cysteine residue, a glutamic acid residue, a glutamine residue, a glycine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is an isoleucine residue, it can be substituted in the modified mature polypeptide by an amino acid which is different from an isoleucine residue, such as, for example, an alanine residue, an arginine residue, an aspartic acid residue, a cysteine residue, a glutamic acid residue, a glutamine residue, a glycine residue, a histidine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a leucine residue, it can be substituted in the modified mature polypeptide by an amino acid which is different from a leucine residue, such as, for example, an alanine residue, an arginine residue, an aspartic acid residue, a cysteine residue, a glutamic acid residue, a glutamine residue, a glycine residue, a histidine residue, an isoleucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a lysine residue, it can be substituted in the modified mature polypeptide by an amino acid which is different from a lysine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a methionine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a methionine residue, it can be substituted in the modified mature polypeptide by an amino acid which is different from a methionine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a phenylalanine residue, a proline residue, a serine residue, a threonine residue, a tryptophan residue, a tyrosine residue, or a valine residue. In an embodiment in which the amino acid residue at position 1 of the control mature polypeptide is a phenylalanine residue, it can be substituted in the modified mature polypeptide by an amino acid which is different from a phenylalanine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a proline residue, a serine residue, a threonine residue, a tryptophan residue, a tyrosine residue, or a valine residue. In an embodiment in which the amino acid residue at position 1 of the control mature polypeptide is a proline residue, it can be substituted in the modified mature polypeptide by an amino acid which is different from a proline residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a serine residue, a threonine residue, a tryptophan residue, a tyrosine residue, or a valine residue. In an embodiment in which the amino acid residue at position 1 of the control mature polypeptide is a proline residue, it can be substituted in the modified mature polypeptide by an amino acid which is different from a proline residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a serine residue, a threonine residue, a tryptophan residue, a tyrosine residue, or a valine residue. In an embodiment in which the amino acid residue at position 1 of the control mature polypeptide is a serine residue, it can be substituted in the modified mature polypeptide by an amino acid which is different from a serine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a threonine residue, a tryptophan residue, a tyrosine residue, or a valine residue. In an embodiment in which the amino acid residue at position 1 of the control mature polypeptide is a serine residue, it can be substituted in the modified mature polypeptide by a glycine residue. In an embodiment in which the amino acid residue at position 1 of the control mature polypeptide is a serine residue, it can be substituted in the modified mature polypeptide by a threonine residue. In an embodiment in which the amino acid residue at position 1 of the control mature polypeptide is a threonine residue, it can be substituted in the modified mature polypeptide by an amino acid which is different from a threonine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a tryptophan residue, a tyrosine residue, or a valine residue. In an embodiment in which the amino acid residue at position 1 of the control mature polypeptide is a tryptophan residue, it can be substituted in the modified mature polypeptide by an amino acid which is different from a tryptophan residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tyrosine residue, or a valine residue. In an embodiment in which the amino acid residue at position 1 of the control mature polypeptide is a tyrosine residue, it can be substituted in the modified mature polypeptide by an amino acid which is different from a tyrosine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, or a valine residue. In an embodiment in which the amino acid residue at position 1 of the control mature polypeptide is a valine residue, it can be substituted in the modified mature polypeptide by an amino acid which is different from a valine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, or a tyrosine residue.

In an embodiment in which the amino acid residue at position 2 of the control mature polypeptide is an alanine residue, it can be substituted in the modified mature polypeptide by an amino acid which is different from an alanine residue, such as, for example, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is an arginine residue, it can be substituted in the modified mature polypeptide by an amino acid which is different from an arginine residue, such as, for example, an alanine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is an asparagine residue, it can be substituted in the modified mature polypeptide by an amino acid which is different from an asparagine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is an aspartic acid residue, it can be substituted in the modified mature polypeptide by an amino acid which is different from an aspartic acid residue, such as, for example, an alanine residue, an arginine residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a cysteine residue, it can be substituted in the modified mature polypeptide by an amino acid which is different from a cysteine residue, such as, for example, an alanine residue, an arginine residue, an aspartic acid residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a glutamic acid residue, it can be substituted in the modified mature polypeptide by an amino acid which is different from a glutamic acid residue, such as, for example, an alanine residue, an arginine residue, an aspartic acid residue, a cysteine residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a glutamine residue, it can be substituted in the modified mature polypeptide by an amino acid which is different from a glutamine residue, such as, for example, an alanine residue, an arginine residue, an aspartic acid residue, a cysteine residue, a glutamic acid residue, a glycine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a glycine residue, it can be substituted in the modified mature polypeptide by an amino acid which is different from a glycine residue, such as, for example, an alanine residue, an arginine residue, an aspartic acid residue, a cysteine residue, a glutamic acid residue, a glutamine residue, a histidine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a histidine residue, it can be substituted in the modified mature polypeptide by an amino acid which is different from a histidine residue, such as, for example, an alanine residue, an arginine residue, an aspartic acid residue, a cysteine residue, a glutamic acid residue, a glutamine residue, a glycine residue, an isoleucine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is an isoleucine residue, it can be substituted in the modified mature polypeptide by an amino acid which is different from an isoleucine residue, such as, for example, an alanine residue, an arginine residue, an aspartic acid residue, a cysteine residue, a glutamic acid residue, a glutamine residue, a glycine residue, a histidine residue, a leucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a leucine residue, it can be substituted in the modified mature polypeptide by an amino acid which is different from a leucine residue, such as, for example, an alanine residue, an arginine residue, an aspartic acid residue, a cysteine residue, a glutamic acid residue, a glutamine residue, a glycine residue, a histidine residue, an isoleucine residue, a lysine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a lysine residue, it can be substituted in the modified mature polypeptide by an amino acid which is different from a lysine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a methionine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a methionine residue, it can be substituted in the modified mature polypeptide by an amino acid which is different from a methionine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a phenylalanine residue, a proline residue, a serine residue, a threonine residue, a tryptophan residue, a tyrosine residue, or a valine residue. In an embodiment in which the amino acid residue at position 2 of the control mature polypeptide is a phenylalanine residue, it can be substituted in the modified mature polypeptide by an amino acid which is different from a phenylalanine residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a proline residue, a serine residue, a threonine residue, a tryptophan residue, a tyrosine residue, or a valine residue. In an embodiment in which the amino acid residue at position 2 of the control mature polypeptide is a proline residue, it can be substituted in the modified mature polypeptide by an amino acid which is different from a proline residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a serine residue, a threonine residue, a tryptophan residue, a tyrosine residue, or a valine residue. In an embodiment in which the amino acid residue at position 2 of the control mature polypeptide is a proline residue, it can be substituted in the modified mature polypeptide by an amino acid which is different from a proline residue, such as, for example, an alanine residue, an arginine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a serine residue, a threonine residue, a tryptophan residue, a tyrosine residue, or a valine residue. In an embodiment in which the amino acid residue at position 2 of the control mature polypeptide is a serine residue, it can be substituted in the modified mature polypeptide by an amino acid which is different from a serine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a threonine residue, a tryptophan residue, a tyrosine residue, or a valine residue. In an embodiment in which the amino acid residue at position 2 of the control mature polypeptide is a serine residue, it can be substituted in the modified mature polypeptide by a glycine residue. In an embodiment in which the amino acid residue at position 2 of the control mature polypeptide is a serine residue, it can be substituted in the modified mature polypeptide by a threonine residue. In an embodiment in which the amino acid residue at position 2 of the control mature polypeptide is a threonine residue, it can be substituted in the modified mature polypeptide by an amino acid which is different from a threonine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a tryptophan residue, a tyrosine residue, or a valine residue. In an embodiment in which the amino acid residue at position 2 of the control mature polypeptide is a tryptophan residue, it can be substituted in the modified mature polypeptide by an amino acid which is different from a tryptophan residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tyrosine residue, or a valine residue. In an embodiment in which the amino acid residue at position 2 of the control mature polypeptide is a tyrosine residue, it can be substituted in the modified mature polypeptide by an amino acid which is different from a tyrosine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, or a valine residue. In an embodiment in which the amino acid residue at position 2 of the control mature polypeptide is a valine residue, it can be substituted in the modified mature polypeptide by an amino acid which is different from a valine residue, such as, for example, an alanine residue, an arginine residue, an asparagine residue, an aspartic acid residue, a cysteine residue, a glutamic acid residue, a glutamine residue, a glycine residue, a histidine residue, an isoleucine residue, a leucine residue, a lysine residue, a methionine residue, a phenylalanine residue, a proline residue, a serine residue, a threonine residue, a tryptophan residue, or a tyrosine residue.

The methods described herein can be used to increase the thermodynamic stability and/or the kinetic stability of the modified mature polypeptide, when compared to the control mature polypeptide. In embodiments in which the modified/control mature polypeptide is an enzyme, the methods described herein can be used to increase the specific enzyme activity (e.g., the number of enzyme units/vol by the concentration of the protein in weight/vol).

In some embodiments, the method can comprise expressing the modified mature polypeptide in the intracellular form in the recombinant eukaryotic host cell. In additional embodiments, the method can further comprises making compositions, fractions and/or preparations comprising the modified mature polypeptide.

The method is especially suited for optimizing control mature polypeptide which have been previously expressed by a prokaryotic host cell (in an intracellular or in a secreted form) or by a eukaryotic host cell (in a secreted form). Without wishing to bound to theory, polypeptides which have been previously expressed by a prokaryotic host cell (in an intracellular or in a secreted form) or by a eukaryotic host cell (in a secreted form) are usually not submitted to N-terminal acetylation. Such polypeptides, if and when expressed by a eukaryotic host cell in an intracellular form, are more susceptible of being submitted to N-terminal acetylation. It is possible that the presence of N-terminal acetylation decreases the stability and/or the biological activity of the control mature polypeptide. This can be observed especially when the parental mature polypeptide is in a globular form and/or its N-terminus interacts with its C-terminus, as the presence of the acetyl residue at the N-terminus may destabilize the formation and maintenance of the globular form and/or the interaction between the N-terminus and the C-terminus. As such, by modifying the amino acid sequence of the control mature polypeptide to limit or reduce N-terminal acetylation it may be possible to increase the stability and/or the biological activity of the modified mature polypeptide.

In some embodiments, the method is performed on control mature polypeptides which have been previously characterized as having a ratio of non-N-terminally acetylated to N-terminally acetylated equal to or below 1.0. In some further embodiments, the method can include a step of determining the ratio of non-N-terminally acetylated to N-terminally acetylated control mature polypeptides which have been expressed in an intracellular form in a recombinant eukaryotic host cell. In some embodiments, the method is performed on control mature polypeptides which have been previously characterized as having a ratio of N-terminally acetylated non-N-terminally acetylated to equal to or higher than 1.0. In some further embodiments, the method can include a step of determining the ratio of N-terminally acetylated to non-N-terminally acetylated control mature polypeptides which have been expressed in an intracellular form in a recombinant eukaryotic host cell.

In some embodiments, the method can be used to increase the ratio of non-N-terminally acetylated to N-terminally acetylated equal to or below 1.0 in modified mature polypeptides (when compared to control mature polypeptides). In some further embodiments, the method can include a step of determining the ratio of non-N-terminally acetylated to N-terminally acetylated modified mature polypeptides which have been expressed in an intracellular form in a recombinant eukaryotic host cell. In some embodiments, the method is to decrease the ratio of N-terminally acetylated non-N-terminally acetylated to equal to or higher than 1.0 in modified mature polypeptides (when compared to control mature polypeptides). In some further embodiments, the method can include a step of determining the ratio of N-terminally acetylated to non-N-terminally acetylated modified mature polypeptides which have been expressed in an intracellular form in a recombinant eukaryotic host cell.

The present invention will be more readily understood by referring to the following examples which are given to illustrate the invention rather than to limit its scope.

### EXAMPLE

Maltogenic alpha-amylase secreted by Bacillus subtilis. A commercial preparation of a maltogenic alpha-amylase having the amino acid sequence of SEQ ID NO: 2 secreted by Bacillus sp. was used.

Maltogenic alpha-amylases produced by Saccharomyces cerevisiae. Various strains of Saccharomyces cerevisiae were engineered to each express a different maltogenic alpha-amylase (see Table 1 for a description of the strains and Table 2 for the amino acid sequence of the different maltogenic amylases expressed). The S. cerevisiae strains were propagated, submitted to autolysis and the maltogenic alpha-amylase and the supernatant was separated from the insoluble cellular debris prior to characterization.

TABLE 1
Saccharomyces cerevisiae strains and isolates used in this Example.
The various strains were obtained from
modifying parental strain M10474.

| | Nucleic acid sequence | Amino acid sequence |
|---|---|---|
| | of the heterologous | encoded by the |
| | nucleic acid | heterologous nucleic |
| Strain or isolate | molecule(s) introduced | acid molecule(s) |
| M16168 | SEQ ID NO: 12 | SEQ ID NO: 2 |
| M17906 | SEQ ID NO: 13 | SEQ ID NO: 3 |
| M30093 | SEQ ID NO: 14 | SEQ ID NO: 5 |
| 1-12H | SEQ ID NO: 15 | SEQ ID NO: 6 |
| M30112 | SEQ ID NO: 10 | SEQ ID NO: 7 |
| | SEQ ID NO: 11 | SEQ ID NO: 9 |
| 2-7A | SEQ ID NO: 16 | SEQ ID NO: 8 |

TABLE 2
Amino acid sequence of the maltogenic alpha-amylases
used in this Example.

| Designation | Amino acid sequence |
|---|---|
| Wild-type | SEQ ID NO: 2 |
| Variant 1 | SEQ ID NO: 3 |
| Variant 2 | SEQ ID NO: 5 |
| Variant 3 | SEQ ID NO: 6 |
| Variant 4 | SEQ ID NO: 7 and 9 |
| Variant 5 | SEQ ID NO: 8 |

Thermostability assessment. Enzyme samples were heated to 25° C. or 85° C. for 5 min at pH 5.0 before adding the 0.5% raw starch (FIG. 1) or 15 mM p-nitrophenol-maltoside (FIGS. 2 and 3). The hydrolysis activity was detected by monitoring the reducing sugar (using a DNS (3,5-dinitrosalicylic acid) assay, FIG. 1) or 4-nitrophenol (by monitoring absorbance at 405 nm, FIGS. 2 and 3) released from the substrate. The hydrolysis activity was compared to the one obtained prior to the heat treatment to calculate the percentage of residual activity.

Melting temperature determination. Melting temperatures (as provided in Table 2) were determined by a thermal shift assay using the SYPRO™ Orange Fluorescent Dye (Differential Scanning Fluorimetry).

Sourdough bread making and assessment. Crumb softening effects of maltogenic amylase enzymes and variants were assessed with bake tests generating open top sour dough breads. Part of the flour was fermented overnight with lactic acid bacteria culture (to pH 4.0-4.5) and was used to make the final sourdough bread the next day. The evaluation of the crumb softness and resilience was done with the TA.XTPlus™ texture analyzer. Two slices of bread of 2.5 cm thickness were used for the test. Five measurements per slice were made on two slices of the middle of the bread for a total of 10 measurements. A macro was used for the calculations of the % resilience.

Sandwich bread marking and assessment. Sandwich bread bake tests were conducted to compare different enzymes variants for their crumb softening effects. A regular bread making process was used. The evaluation of the crumb softness and resilience was done with the TA.XTPlus™ texture analyzer. Two slices of bread of 2.5 cm thickness were used for the test. Five measurements per slice were made on two slices of the middle of the bread for a total of 10 measurements. A macro was used for the calculations of the % resilience.

Sweet dough bread making and assessment. Crumb softening effects of maltogenic amylase enzymes and variants were assessed with bake tests generating 25% sugar dough breads. The bread making process was similar to the sandwich bread making process, but a higher sugar level was included in the dough (25% sucrose). The evaluation of the crumb softness and resilience was done with the TA.XTP/us™ texture analyzer. Two slices of bread of 2.5 cm thickness were used for the test. Five measurements per slice were made on two slices of the middle of the bread for a total of 10 measurements. A macro was used for the calculations of the % resilience.

Acetylation Determination. The Acetylation of Polypeptides was Determined by Mass Spectrometry.

Optimal pH determination. Samples were obtained from an ultrafiltration retentate obtained from the culture of strain M30112. Aliquots of the sample were diluted into Mcllvaine buffer preparations with pH values ranging from 3 to 8. Ten (10) μL of the enzyme dilutions at different pH values was then mixed with 50 μL of 1% wheat starch. The reactions were then incubated at 60° C. for 10 minutes. Reactions were stopped through incubation with 100 μL DNS (1% w/v 3,5-dinitrosalicylic acid, 1% w/v sodium hydroxide, 0.05% w/v sodium sulfide) for 5 minutes at 99° C. The absorbance at 540 nm was then measured spectrophotometrically. The magnitude of the A540 reading is proportional with the concentration of released maltose in the sample, and consequently to the enzymatic activity.

Optimal temperature determination. Samples were obtained from an ultrafiltration retentate obtained from the culture of strain M30112. Aliquots of the sample were diluted into Mcllvaine buffer at pH 5. A set of 12 identical reactions were prepared by mixing 10 μL of the enzyme dilutions with 50 μL 1% wheat starch slurry, and incubated at 12 different temperatures (45-100° C., 5° C. increments) for 10 minutes. Each reaction was performed with three technical replicates. The end point absorbance at 540 nm was measured to quantify the reaction, as described in the section “Optimal pH determination”.

The maltogenic amylase from Geobacillus stearothermophilus (Uniprot P19531, which, in a secreted (mature) form, corresponds to the amino acid sequence of SEQ ID NO: 2) is an enzyme that has been used as a bread improvement enzyme for anti-staling effects. This enzyme has been previously expressed in a secreted form, in a recombinant Bacillus subtilis. It was however sought to express the enzyme (having the amino acid sequence of SEQ ID NO: 2) in an intracellular form using a recombinant Saccharomyces cerevisiae host cell. However, when the enzyme of SEQ ID NO: 2 was expressed intracellularly in S. cerevisiae, it exhibited a lowered thermostability, when compared to the same enzyme secreted by a recombinant Bacillus subtilis host cell (FIG. 1, Table 3). This lowered thermostability lead to significantly lowered performance of this enzyme in baking applications (data not shown).

TABLE 3
Melting temperature determined by thermal shift assays for
the maltogenic alpha-amylase having the amino acid sequence of
SEQ ID NO: 2 either expressed G. intracellularly
by S. cerevisiae and secreted by B. subtilis.

| | pH 5.5, | pH 5.5, |
|---|---|---|
| | without NaCl | with 100 mM |
| Enzyme variant | (° C.) | NaCl (° C.) |
| Wild-type (secreted from B. subtilis) | 81.9 | 83.9 |
| Wild-type (expressed intracellularly | 80.6 | 81.4 |
| in S. cerevisiae) | | |
| Var 2 (expressed intracellularly | 83.0 | 84.4 |
| in S. cerevisiae) | | |
| Var 1 (expressed intracellularly | 89.3 | 90.0 |
| in S. cerevisiae) | | |
| Var 4 (expressed intracellularly | 90.0 | 91.0 |
| in S. cerevisiae) | | |

To alleviate this negative effect, the N-terminus of the maltogenic amylase was engineered to change its local chemical and physical properties to obtain a globally more stable conformation. A site-saturation mutagenesis of the first residue on the N-terminus of the protein, and isolated two variants that showed improved thermostability: variant 2 (S1G substitution when compared to wild-type) and variant 3 (S1T substitution when compared to wild-type). FIG. 2 shows that the thermostability of variants 2 and 3 was significantly improved over the wild-type enzyme. The effectiveness of variant 2 with a wild-type reference enzyme having optimal performance was compared. The results indicated that, in order to achieve the same softness effectiveness and resilience as the wild-type enzyme, a dose which 8× lower for variant 2 could be used (FIGS. 3A and 3B).

Variant 1 is a thermostable variant of the wild-type enzyme (Jones et al., 2008). Similarly, to what was observed about with variants 2 and 3, variants 4 (S1G substitution when compared to variant 1) and 5 (S1T substitution when compared to variant 1) exhibited improved thermostability when compared to variant 1 when the enzymes were expressed intracellularly in S. cerevisiae (FIG. 4). The use of Variant 4 in the production of sourdough breads resulted in breads having reduced crumb hardness (FIG. 5A) and higher resilience (FIG. 5B) than sourdough breads made with Variant 1. The use of Variant 4 in the production of sweetened breads resulted in breads having reduced crumb hardness (FIG. 6A) and higher resilience (FIG. 6B) than sweetened breads made with Variant 1 or the reference maltogenic alpha-amylase.

The N-terminal acetylation of variants 1 and 4 expressed intracellularly by S. cerevisiae was determined. It was determined that 100% of variants 1 were acetylated at the N-terminus. This was similar to the wild-type enzyme, which was also 100% acetylated at the N-terminus. Interestingly, only 6% of variants 4 have the N-terminus acetylated, and 92% of variants 4 have an unmodified/unacetylated N-terminus. Additionally, 5.6% of variants 2 have the N-terminus acetylated, and 94.4% of variants 2 have an unmodified/unacetylated N-terminus.

As shown on FIG. 7A, the optimal pH of variant 4 was determined to be around 4.5. As shown on FIG. 7B, the optimal temperature of variant 4 was determined to be around 80° C.

### REFERENCES

Jones A, Lamsa M, Frandsen T P, SpendlerT, Harris P, Sloma A, Xu F, Nielsen J B, Cherry J R. Directed evolution of a maltogenic alpha-amylase from Bacillus sp. TS-25. J Biotechnol. 2008 Apr. 30; 134(3-4):325-33.

1. A recombinant eukaryotic host cell capable of expressing a heterologous mature polypeptide in an intracellular form, wherein:
the amino acid sequence of the heterologous mature polypeptide has at least one amino acid modification, when compared to the amino acid sequence of a control mature polypeptide; and
the at least one amino acid modification:
is located within the N-terminal region of the amino acid sequence of the control mature polypeptide; and
increases the stability and/or the biological activity of the heterologous mature polypeptide compared to the control mature polypeptide expressed in an intracellular form by a control recombinant eukaryotic host cell.

2. The recombinant eukaryotic host cell of claim 1, wherein the at least one amino acid modification:
increases the thermodynamic stability and/or the kinetic stability of the heterologous mature polypeptide compared to the control mature polypeptide;
increases the ratio of non-N-terminally acetylated to N-terminally acetylated of the heterologous mature polypeptide, when compared to the control mature polypeptide the recombinant eukaryotic host cell and the control recombinant eukaryotic host cell are capable of performing N-terminal acetylation;
is located at position 1 and/or 2 of the amino acid sequence of the control mature polypeptide; and/or
comprises at least one amino acid substitution.

3.-6. (canceled)

7. The recombinant eukaryotic host cell of claim 1, wherein the heterologous mature polypeptide:
has been hydrolyzed by a methionine aminopeptidase;
is a globular protein; and/or
is an enzyme.

8.-10. (canceled)

11. The recombinant eukaryotic host cell of claim 7, wherein the enzyme is a glycoside hydrolase.

12. The recombinant eukaryotic host cell of claim 11, wherein the glycoside hydrolase is an amylase, a cellulase, or a hemi-cellulase.

13. The recombinant eukaryotic host cell of claim 12, wherein the amylase is a maltogenic alpha-amylase.

14. (canceled)

15. The recombinant eukaryotic host cell of claim 13, wherein the control mature polypeptide has the amino acid sequence of SEQ ID NO: 2 or 3, is a variant of the amino acid sequence of SEQ ID NO: 2 or 3 lacking the at least one amino acid modification and exhibiting maltogenic alpha-amylase activity or is a fragment of the amino acid sequence of SEQ ID NO: 2 or 3 comprising the N-terminal region and exhibiting maltogenic alpha-amylase activity.

16. The recombinant eukaryotic host cell of claim 13, wherein the heterologous mature polypeptide has the amino acid sequence of any one of SEQ ID NO: 4 to 9, is a variant of the amino acid sequence of any one of SEQ ID NO: 4 to 9 having the at least one amino acid modification and exhibiting maltogenic alpha-amylase activity or is a fragment of the amino acid sequence of any one of SEQ ID NO: 4 to 9 comprising the N-terminal region and exhibiting maltogenic alpha-amylase activity.

17. The recombinant eukaryotic host cell of claim 1 being a recombinant yeast host cell.

18. The recombinant eukaryotic host cell of claim 17 being from Saccharomyces sp. or a Komagataella sp.

19. A process for making a heterologous mature polypeptide, the process comprising propagating, and optionally fermenting, the recombinant eukaryotic host cell of claim 1 under conditions allowing the expression and the intracellular accumulation of the heterologous mature polypeptide.

20.-22. (canceled)

23. A combination of maltogenic alpha-amylases comprising: (i) the polypeptide having amino acid sequence of any one of SEQ ID NO: 4 to 9, being a variant of the amino acid sequence of any one of SEQ ID NO: 4 to 9 having the at least one amino acid modification and exhibiting maltogenic alpha-amylase activity or being a fragment of the amino acid sequence of any one of SEQ ID NO: 4 to 9 having the at least one amino acid modification and exhibiting maltogenic alpha-amylase activity and (ii) a further maltogenic alpha-amylase.

24. A heterologous mature polypeptide obtained from a recombinant eukaryotic host cell capable of expressing the heterologous mature polypeptide in an intracellular form, wherein:
the amino acid sequence of the heterologous mature polypeptide has at least one amino acid modification, when compared to the amino acid sequence of a control mature polypeptide; and
the at least one amino acid modification:
is located within the N-terminal region of the amino acid sequence of the control mature polypeptide; and
increases the stability and/or the biological activity of the heterologous mature polypeptide compared to the control mature polypeptide expressed in an intracellular form by a control recombinant eukaryotic host cell.

25.-35. (canceled)

36. The heterologous mature polypeptide of claim 24 having the amino acid sequence of any one of SEQ ID NO: 4 to 9, being a variant of the amino acid sequence of any one of SEQ ID NO: 4 to 9 having the at least one amino acid modification and exhibiting maltogenic alpha-amylase activity or being a fragment of the amino acid sequence of any one of SEQ ID NO: 4 to 9 having the N-terminal region and exhibiting maltogenic alpha-amylase activity.

37. A process for preparing a dough or a baked product prepared from the dough, the process comprising adding an effective amount of the heterologous mature polypeptide of claim 36, optionally in combination with a fermenting yeast, to the dough.

38.-49. (canceled)
